(19) United States
(12) Patent Application Publication
(10) Pub. No.: US 2020/0368280 A1
(43) Pub. Date: Nov. 26, 2020

(54) EPIGENETIC MODIFIERS FOR USE IN CELLULAR IMMUNOTHERAPY

(71) Applicant: Viracta Therapeutics, Inc., Cardiff, CA (US)

(72) Inventors: Marshelle WARREN, Cardiff, CA (US); Preston DANIELS, Solana Beach, CA (US)

(21) Appl. No.: 16/961,200

(22) PCT Filed: Jan. 11, 2019
(86) PCT No.: PCT/US2019/013343

§ 371 Date: Jul. 9, 2020

Related U.S. Application Data
(60) Provisional application No. 62/616,791, filed on Jan. 12, 2018.
(60) Provisional application No. 62/618,455, filed on Jan. 17, 2018.

(51) Int. Cl. A61K 35/17; A61P 35/02; A61K 31/506; A61K 39/395; A61P 31/18; C12N 5/0783

(52) U.S. Cl. CPC A61K 35/17; A61P 35/02; A61K 31/506; A61K 2039/505; A61P 31/18; C12N 5/0636; A61K 39/3955

(57) ABSTRACT

Described herein are methods and compositions useful for augmenting cell-based immunotherapies. The augmented cell-based immunotherapies can be used to treat individuals with cancer and chronic viral infections.

### CROSS REFERENCE TO RELATED APPLICATIONS

This application claims the benefit of U.S. Provisional Application Ser. No. 62/616,791 filed on Jan. 12, 2018 and U.S. Provisional Application Ser. No. 62/618,455 filed on Jan. 17, 2018, both of which are incorporated by reference herein in their entirety.

### BACKGROUND OF THE INVENTION

Immunotherapy is an emerging method for the treatment of cancer and chronic viral diseases. Immunotherapy is based upon using constituents of the immune system either molecular or cellular. Molecular therapies include recombinant cytokines, chemokines, antibodies, and other immune modulating polypeptides, proteins, or small molecules. Cellular based therapies include, administering lymphocyte populations, such as, antigen presenting cells, NK cells, or T cells to modulate a patient's immune response and direct it to eliminating a chronic viral infection, a malignancy, or a tumor.

### SUMMARY OF THE INVENTION

Exhaustion is a hallmark of, and obstacle to, many cell-based immunotherapies. Exhaustion is the decreased functionality and effectiveness of an immune effector cell's response to specific antigen. In individuals with cancer or chronic viral infections antigen specific T cells are generally present, yet when exhausted, lack the ability to proliferate, secrete helper cytokines/chemokines, or kill target cells that display antigen. Exhaustion effects both CD4+ and CD8+ T cells. Other cells that are deployed in cell based therapies, such as NK cells, can exhibit signs of exhaustion marked by decreases in cytokine secretion and target cell killing. Generally, exhausted immune effector cells display epigenetic differences when compared to a non-exhausted cell. Therefore, treating an exhausted T cell or NK cell with the proper HDAC inhibitor (HDACi) will reverse T-cell exhaustion and augment a cell-based immunotherapy. Described herein are methods of deploying the HDACi nanatinostat in conjunction with cell-based immunotherapies, therefore enhancing the therapies and their uses to treat diseases associated with immune cell exhaustion.

Described herein are methods to augment cell-based immunotherapies using an HDACi. The HDAC inhibitors for use in augmenting the immunotherapies described herein display unexpectedly superior results and potency compared to other HDAC inhibitors. In certain embodiments, the HDACi inhibit deacetylation of histone H3. (e.g., increase steady-state acetylation of Histone H3). In various embodiments, these HDACi can be deployed in vitro to treat a lymphocyte population (e.g., T cells NK cells) to be used in an adoptive cell therapy. In certain instances, a patient's own cells can be treated in vitro before re-administration to the same patient. In other embodiments, a primary cell population or a cell line that is not isolated from a patient being treated can be treated in vitro. In certain embodiments, cells from an HLA matched donor can be treated with the HDACi. In certain embodiments, cells from an HLA mismatched donor or cell line can be treated with the HDACi. In certain specific embodiments, the HDACi is nanatinostat (2-(6-{[(6-Fluoroquinolin-2-yl)methyl]amino}-3-azabicyclo[3.1.0]hex-3-yl)-N-hydroxypyrimidine-5-carboxamide).

In a certain aspect, described herein, is a method for augmenting a cell-based immunotherapy comprising contacting a cell-based immunotherapy in vitro with an HDAC inhibitor (HDACi), wherein the HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide). In certain embodiments, the method reverses T cell exhaustion. In certain embodiments, the concentration of the HDACi is an amount sufficient to increase acetylation of histone H3. In certain embodiments, the concentration of the HDACi is less than about 1 micromolar. In certain embodiments, the concentration of the HDACi is greater than about 400 nanomolar. In certain embodiments, the HDACi is contacted with the cell-based immunotherapy for at least 2 hours. In certain embodiments, the HDACi is contacted with the cell-based immunotherapy for at least 16 hours. In certain embodiments, the method comprises contacting the cell-based immunotherapy with interleukin-15. In certain embodiments, the interleukin-15 is contacted with the cell-based immunotherapy at a concentration of about 1 to about 100 ng/mL. In certain embodiments, the interleukin-15 is contacted with the cell-based immunotherapy at a concentration of about 5 to about 25 ng/mL. In certain embodiments, the interleukin-15 is contacted with the cell-based immunotherapy at a concentration of about 10 ng/mL. In certain embodiments, the method comprises contacting the cell-based immunotherapy with a checkpoint inhibitor. In certain embodiments, the checkpoint inhibitor is an antibody that targets PDL-1 or PD-1. In certain embodiments, the cell-based immunotherapy comprises a T-cell population. In certain embodiments, the T-cell population comprises a primary T-cell population derived from a healthy individual. In certain embodiments, the T-cell population comprises a primary T-cell population derived from an individual afflicted with a disease. In certain embodiments, the T-cell population further comprises a chimeric antigen receptor (CAR). In certain embodiments, the method further comprises stimulating the T-cell population with a tumor associated antigen. In certain embodiments, the method further comprises stimulating the T-cell population with a pro-inflammatory cytokine. In certain embodiments, the T-cell population is enriched for CD4 positive T cells. In certain embodiments, the T-cell population is enriched for CD8 positive T cells. In certain embodiments, FoxP3 expression is reduced in the T-cell population after contacting the cell-based immunotherapy with an HDACi. In certain embodiments, secretion of interferon gamma is increased in the T-cell population after contacting the cell-based immunotherapy with an HDACi. In certain embodiments, cell-surface expression of CXCR3 is increased in the T-cell population after contacting the cell-based immunotherapy with an HDACi. In certain embodiments, the cell-based therapy comprises a T-cell line. In certain embodiments, the T cell line comprises a chimeric antigen receptor. In certain embodiments, FoxP3 expression is reduced in the T cell line after contacting the cell-based immunotherapy with an HDACi. In certain embodiments, secretion of interferon gamma is increased in the T cell line after contacting the cell-based immunotherapy with an HDACi. In certain embodiments, cell-surface expression of CXCR3 is increased in the T cell line after contacting the cell-based immunotherapy with an HDACi. In certain embodiments, the cell-based immunotherapy comprises a primary natural killer cell population. In certain embodiments, the cell-based immunotherapy comprises a natural killer cell line. In certain embodiments, the natural killer cell line or population comprises a chimeric antigen receptor. In certain embodiments, the natural killer cell line or population comprises a high-affinity Fc receptor. In certain embodiments, secretion of interferon gamma is increased in the natural killer cell line or population after contacting the cell-based immunotherapy with an HDACi. In certain embodiments, the method further comprises administering the cell-based immunotherapy to an individual afflicted with a disease. In certain embodiments, the cell-based immunotherapy is autologous to the individual afflicted with a disease. In certain embodiments, the disease is a cancer. In certain embodiments, the cancer is breast cancer, cervical cancer, colon cancer, head and neck cancer, kidney cancer, liver cancer, lung cancer, melanoma, ovarian cancer, pancreatic cancer, or prostate cancer. In certain embodiments, the cancer is a leukemia or lymphoma. In certain embodiments, the disease is a chronic viral disease. In certain embodiments, the chronic viral disease is caused by the human immunodeficiency virus, human cytomegalovirus, Epstein-Barr virus, hepatitis C virus, hepatitis B virus, or human papilloma virus (HPV).

In another aspect, described herein, is a method of adoptive cell immunotherapy comprising: a) contacting a cell-based immunotherapy with an HDAC inhibitor (HDACi), wherein the HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide); and b) administering the cell-based immunotherapy to an individual afflicted with a disease. In certain embodiments, contacting the cell-based immunotherapy with the HDACi is performed in vitro. In certain embodiments, the concentration of the HDACi is an amount sufficient to increase acetylation of histone H3. In certain embodiments, the concentration of the HDACi is less than about 1 micromolar. In certain embodiments, the HDACi is contacted with the cell-based immunotherapy for at least 2 hours. In certain embodiments, the HDACi is contacted with the cell-based immunotherapy for at least 16 hours. In certain embodiments, the method comprises contacting the cell-based immunotherapy with interleukin-15. In certain embodiments, the interleukin-15 is contacted with the cell-based immunotherapy at a concentration of about 1 to about 100 ng/mL. In certain embodiments, the interleukin-15 is contacted with the cell-based immunotherapy at a concentration of about 5 to about 25 ng/mL. In certain embodiments, the interleukin-15 is contacted with the cell-based immunotherapy at a concentration of about 10 ng/mL. In certain embodiments, the method comprises contacting the cell-based immunotherapy with a checkpoint inhibitor. In certain embodiments, the checkpoint inhibitor is an antibody that targets PDL-1 or PD-1. In certain embodiments, the cell-based immunotherapy comprises a T-cell population. In certain embodiments, the T-cell population comprises a primary T-cell population derived from a healthy individual. In certain embodiments, the T-cell population comprises a primary T-cell population derived from an individual afflicted with a disease. In certain embodiments, the T-cell comprises a primary T-cell population derived from the individual afflicted with the disease. In certain embodiments, the T-cell population further comprises a chimeric antigen receptor (CAR). In certain embodiments, the method further comprises stimulating the T-cell population with a tumor associated antigen. In certain embodiments, the method further comprises stimulating the T-cell population with a pro-inflammatory cytokine. In certain embodiments, the T-cell population is enriched for CD4 positive T cells. In certain embodiments, the T-cell population is enriched for CD8 positive T cells. In certain embodiments, FoxP3 expression is reduced in the T-cell population after contacting the cell-based immunotherapy with the HDACi. In certain embodiments, secretion of interferon gamma is increased in the T-cell population after contacting the cell-based immunotherapy with the HDACi. In certain embodiments, cell-surface expression of CXCR3 is increased in the T-cell population after contacting the cell-based immunotherapy with the HDACi. In certain embodiments, the cell-based immunotherapy comprises a T-cell line. In certain embodiments, the T cell line comprises a chimeric antigen receptor. In certain embodiments, FoxP3 expression is reduced in the T cell line after contacting the cell-based immunotherapy with an HDACi. In certain embodiments, secretion of interferon gamma is increased in the T cell line after contacting the cell-based immunotherapy with an HDACi. In certain embodiments, cell-surface expression of CXCR3 is increased in the T cell line after contacting the cell-based immunotherapy with an HDACi. In certain embodiments, the cell-based immunotherapy comprises a primary natural killer cell population. In certain embodiments, the cell-based immunotherapy comprises a natural killer cell line. In certain embodiments, the natural killer cell line or population comprises a chimeric antigen receptor. In certain embodiments, the natural killer cell line or population comprises a high-affinity Fc receptor. In certain embodiments, secretion of interferon gamma is increased in the natural killer cell line or population after contacting the cell-based immunotherapy with an HDACi. In certain embodiments, the disease is a cancer. In certain embodiments, the cancer is breast cancer, cervical cancer, colon cancer, head and neck cancer, kidney cancer, liver cancer, lung cancer, melanoma, ovarian cancer, pancreatic cancer, or prostate cancer. In certain embodiments, the cancer is a leukemia or lymphoma. In certain embodiments, the disease is a chronic viral disease. In certain embodiments, the chronic viral disease is caused by the human immunodeficiency virus, human cytomegalovirus, Epstein-Barr virus, hepatitis C virus, or hepatitis B virus, or human papilloma virus (HPV).

In another aspect, described herein, is a cell culture media comprising an HDAC inhibitor (HDACi), wherein the HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide). In certain embodiments, the cell culture media does not comprise serum of non-human origin. In certain embodiments, the cell culture media does not comprise serum. In certain embodiments, the cell culture media comprises contacting the cell-based immunotherapy with interleukin-15. In certain embodiments, the interleukin-15 is contacted with the cell-based immunotherapy at a concentration of about 1 to about 100 ng/mL. In certain embodiments, the interleukin-15 is contacted with the cell-based immunotherapy at a concentration of about 5 to about 25 ng/mL. In certain embodiments, the interleukin-15 is contacted with the cell-based immunotherapy at a concentration of about 10 ng/mL. In certain embodiments, the cell culture media comprises contacting the cell-based immunotherapy with a checkpoint inhibitor. In certain embodiments, the checkpoint inhibitor is an antibody that targets PDL-1 or PD-1. In certain embodiments, the cell culture media further comprises a cell-based immunotherapy. In certain embodiments, the cell-based immunotherapy comprises a T-cell population. In certain embodiments, the T-cell population comprises a primary T-cell population derived from a healthy individual. In certain embodiments, the T-cell population comprises a primary T-cell population derived from an individual afflicted with a disease. In certain embodiments, the T-cell population comprises a primary T-cell population derived from the individual afflicted with the disease. In certain embodiments, the T-cell population further comprises a chimeric antigen receptor (CAR). In certain embodiments, the cell culture media further comprises a tumor associated antigen. In certain embodiments, the cell culture media further comprises a pro-inflammatory cytokine. In certain embodiments, the T-cell population is enriched for CD4 positive T cells. In certain embodiments, the T-cell population is enriched for CD8 positive T cells. In certain embodiments, FoxP3 expression is reduced in the T-cell population after contacting the cell-based immunotherapy with the cell culture media. In certain embodiments, secretion of interferon gamma is increased in the T-cell population after contacting the cell-based immunotherapy with the cell culture media. In certain embodiments, cell-surface expression of CXCR3 is increased in the T cell-population after contacting the cell-based immunotherapy with the cell culture media. In certain embodiments, the cell-based immunotherapy comprises a T-cell line. In certain embodiments, the T cell line comprises a chimeric antigen receptor. In certain embodiments, FoxP3 expression is reduced in the T cell line after contacting the cell-based immunotherapy with the cell culture media. In certain embodiments, secretion of interferon gamma is increased in the T cell line after contacting the cell-based immunotherapy with the cell culture media. In certain embodiments, cell-surface expression of CXCR3 is increased in the T cell line after contacting the cell-based immunotherapy with the cell culture media. In certain embodiments, the cell-based therapy comprises a natural killer cell line or primary natural killer cell population. In certain embodiments, the natural killer cell line or population comprises a chimeric antigen receptor. In certain embodiments, the natural killer cell line or population comprises a high-affinity Fc receptor. In certain embodiments, secretion of interferon gamma is increased in the natural killer cell line after contacting the cell-based immunotherapy with the cell culture media. In certain embodiments, the media is for use in a method of inhibiting or reversing T cell exhaustion. In certain embodiments, the media is for use in a method of treating an individual afflicted with a disease. In certain embodiments, the disease is a cancer. In certain embodiments, the cancer is breast cancer, cervical cancer, colon cancer, head and neck cancer, kidney cancer, liver cancer, lung cancer, melanoma, ovarian cancer, pancreatic cancer, or prostate cancer. In certain embodiments, the cancer is a leukemia or lymphoma. In certain embodiments, the disease is a chronic viral disease. In certain embodiments, the chronic viral disease is caused by the human immunodeficiency virus, human cytomegalovirus, Epstein-Barr virus, hepatitis C virus, or hepatitis B virus, or human papilloma virus (HPV).

In another aspect, described herein, is a method of treating human immunodeficiency (HIV) infection in an individual comprising: administering to an individual with an HIV infection an effective amount of nanatinostat, wherein the individual with an HIV infection has an HIV viral load of less than 1000 copies of HIV RNA per milliliter of blood. In certain embodiments, the individual has an HIV viral load of less than 100 copies of HIV RNA per milliliter. In certain embodiments, nanatinostat is administered at a dose of less than 80 mg per day. In certain embodiments, nanatinostat is administered at a dose of less than 40 mg per day. In certain embodiments, nanatinostat is administered at a dose of less than 20 mg per day. In certain embodiments, the method further comprises administering an anti-HIV treatment to the individual with an HIV infection. In certain embodiments, the method further comprises administering an anti-HIV treatment to the individual with an HIV infection. In certain embodiments, the anti-HIV treatment comprises an anti-retroviral drug or pharmaceutically acceptable salt thereof. In certain embodiments, the anti-retroviral drug or pharmaceutically acceptable salt thereof is selected form the list consisting of Abacavir, Atazanavir, Darunavir, Dolutegravir, Efavirenz, Elvitegravir, Emtricitabine, Etravirine, Fosamprenavir, Lamivudine, Lopinavir, Maraviroc, Nevirapine, Raltegravir, Rilpivirine, Ritonavir, Tenofovir, Zidovudine, and combinations thereof. In certain embodiments, the anti-HIV treatment comprises an immunotherapy. In certain embodiments, the immunotherapy comprises an antibody that binds to an HIV derived polypeptide. In certain embodiments, the immunotherapy comprises a T-cell population. In certain embodiments, the T-cell population is transgenic for a chimeric antigen receptor specific for an HIV derived polypeptide. In certain embodiments, the T-cell population is a cytotoxic T cell population that specifically lyses HIV infected cells. In certain embodiments, the immunotherapy comprises a natural killer cell population. In certain embodiments, the natural killer cell population is transgenic for a chimeric antigen receptor specific for an HIV derived polypeptide. In certain embodiments, the immunotherapy is contacted with a histone deacetylase inhibitor (HDACi) in vitro prior to administration to the individual with an HIV infection. In certain embodiments, the HDACi comprises nanatinostat, quisinostat (JNJ-26481585 (N-hydroxy-2-(4-((((1-methyl-1H-indol-3-yl)methyl)amino)methyl)piperidin-1-yl)pyrimidine-5-carboxamide)), R306465/JNJ-16241199 (N-hydroxy-5-(4-(naphthalen-2-ylsulfonyl)piperazin-1-yl)pyrimidine-2-carboxamide), Belinostat/PXD101, trichostatin A/TSA (7-[4-(dimethylamino)phenyl]-N-hydroxy-4,6-dimethyl-7-oxohepta-2,4-dienamide), ITF2357, CBHA, Givinostat/ITF2357, romidepsin, PCI-24781, depsipeptide (FR901228 or FK228), butyrate, phenylbutyrate, valproic acid, AN-9, CI-994, Entinostat/MS-275/SNDX-275, mocetinostat/MGCD0103 (N-(2-aminophenyl)-4-((4-pyridin-3-ylpyrimidin-2-ylamino)methyl)benzamide), m-carboxycinnamic acid, bishydroxamic acid, suberic bishydroxamic acid, oxamflatin, ABHA, SB-55629, pyroxamide, propenamides, aroyl pyrrolyl hydroxamides, or LAQ824 (((E)-N-hydroxy-3-[4-[[2-hydroxyethyl-[2-(1H-indol-3-yl)ethyl]amino]methyl]phenyl]prop-2-enamide), panobinostat/LBH-589, vorinsotat/SAHA, chidamide, or 4SC-202. In certain embodiments, the HDACi comprises nanatinostat. In certain embodiments, the concentration of the HDACi is an amount sufficient to increase acetylation of histone H3. In certain embodiments, the concentration of the HDACi is less than about 1 micromolar. In certain embodiments, the HDACi is contacted with the immunotherapy for at least 2 hours. In certain embodiments, the HDACi is contacted with the immunotherapy for at least 16 hours. In certain embodiments, the individual with an HIV infection has previously received an anti-HIV treatment. In certain embodiments, the anti-HIV treatment is an anti-retroviral drug or pharmaceutically acceptable salt thereof.

In another aspect, described herein, is a method of treating human immunodeficiency (HIV) infection in an individual comprising: administering to an individual with an HIV infection an effective amount of a histone deacetylase inhibitor(HDACi), wherein the HDACi comprises nanatinostat, quisinostat (JNJ-26481585 (N-hydroxy-2-(4-((((1-methyl-1H-indol-3-yl)methyl)amino)methyl)piperidin-1-yl)pyrimidine-5-carboxamide)), R306465/JNJ-16241199 (N-hydroxy-5-(4-(naphthalen-2-ylsulfonyl)piperazin-1-yl)pyrimidine-2-carboxamide), Belinostat/PXD101, trichostatin A/TSA (7-[4-(dimethylamino)phenyl]-N-hydroxy-4,6-dimethyl-7-oxohepta-2,4-dienamide), ITF2357, CBHA, Givinostat/ITF2357, romidepsin, PCI-24781, depsipeptide (FR901228 or FK228), butyrate, phenylbutyrate, valproic acid, AN-9, CI-994, Entinostat/MS-275/SNDX-275, mocetinostat/MGCD0103 (N-(2-aminophenyl)-4-((4-pyridin-3-ylpyrimidin-2-ylamino)methyl)benzamide), m-carboxycinnamic acid, bishydroxamic acid, suberic bishydroxamic acid, oxamflatin, ABHA, SB-55629, pyroxamide, propenamides, aroyl pyrrolyl hydroxamides, or LAQ824 (((E)-N-hydroxy-3-[4-[[2-hydroxyethyl-[2-(1H-indol-3-yl)ethyl]amino]methyl]phenyl]prop-2-enamide), panobinostat/LBH-589, vorinsotat/SAHA, chidamide, or 4SC-202, wherein the individual with an HIV infection has an HIV viral load of less than 1000 copies of HIV RNA per milliliter of blood. In certain embodiments, the individual has an HIV viral load of less than 100 copies of HIV RNA per milliliter. In certain embodiments, the HDACi is administered at a dose of less than 80 mg per day. In certain embodiments, the HDACi is administered at a dose of less than 40 mg per day. In certain embodiments, the HDACi is administered at a dose of less than 20 mg per day. In certain embodiments, the method further comprises administering an anti-HIV treatment to the individual with an HIV infection. In certain embodiments, the anti-HIV treatment comprises an anti-retroviral drug or pharmaceutically acceptable salt thereof. In certain embodiments, the anti-retroviral drug or pharmaceutically acceptable salt thereof is selected form the list consisting of Abacavir, Atazanavir, Darunavir, Dolutegravir, Efavirenz, Elvitegravir, Emtricitabine, Etravirine, Fosamprenavir, Lamivudine, Lopinavir, Maraviroc, Nevirapine, Raltegravir, Rilpivirine, Ritonavir, Tenofovir, Zidovudine, and combinations thereof. In certain embodiments, the anti-HIV treatment comprises an immunotherapy. In certain embodiments, the immunotherapy comprises an antibody that binds to an HIV derived polypeptide. In certain embodiments, the immunotherapy comprises a T-cell population. In certain embodiments, the T-cell population is transgenic for a chimeric antigen receptor specific for an HIV derived polypeptide. In certain embodiments, the T-cell population is a cytotoxic T cell population that specifically lyses HIV infected cells. In certain embodiments, the immunotherapy comprises a natural killer cell population. In certain embodiments, the natural killer cell population is transgenic for a chimeric antigen receptor specific for an HIV derived polypeptide. In certain embodiments, the immunotherapy is contacted with nanatinostat in vitro prior to administration to the individual with an HIV infection. In certain embodiments, the concentration of nanatinostat is an amount sufficient to increase acetylation of histone H3. In certain embodiments, the concentration of nanatinostat is less than about 1 micromolar. In certain embodiments, nanatinostat is contacted with the immunotherapy for at least 2 hours. In certain embodiments, nanatinostat is contacted with the immunotherapy for at least 16 hours. In certain embodiments, the individual with an HIV infection has previously received an anti-HIV treatment. In certain embodiments, the anti-HIV treatment is an anti-retroviral drug or pharmaceutically acceptable salt thereof.

In another aspect, described herein, is a method for treating an individual with a latent viral infection comprising: a) administering to an individual with the latent viral infection a first histone deactylase inhibitor (HDACi); b) contacting a cell-based immunotherapy in vitro with a second HDACi, wherein the second HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide); and c) administering the cell-based immunotherapy to individual with the latent viral infection.

In another aspect, described herein, is a method for treating an individual with a latent viral infection comprising: a) administering to an individual with the latent viral infection a first histone deactylase inhibitor (HDACi), wherein the first HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide)); b) contacting a cell-based immunotherapy in vitro with a second HDACi; and c) administering the cell-based immunotherapy to the individual with the latent viral infection.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings.

FIG. 1A shows quantified FACs data (percentage CD4+, CD25+, FoxP3+) from BALB/c splenocytes treated with Entinostat (1 μM) or nanatinostat 1 μM, 500 nM, 100 nM, 1 nM).

FIG. 1B shows quantified FACs data from BALB/c splenocytes treated with nanatinostat at 1 μM, 500 nM, or 100 nM.

FIG. 2 shows mean tumor volume for mice inoculated with CT26 tumor cell lines and treated with a combination of anti-PD-1 and nanatinostat.

FIGS. 3A and 3B shows mean tumor volume for mice inoculated with 4T1 tumor cell lines and treated with a combination of anti-PD-1 and nanatinostat.

FIG. 3A shows mice treated with 10 mg/kg of nanatinostat and 10 mg/kg anti-PD-1 (filled shapes).

FIG. 3B shows mice treated with 25 mg/kg of nanatinostat and 10 mg/kg anti-PD-1 (filled shapes).

FIG. 4 shows the percentage of CD8+ T cells in tumors of mice treated as indicated.

FIG. 5A shows the percentage of CD4+/CXCR3+ T cells in tumors of mice treated as indicated.

FIG. 5B shows the percentage of CD8+/CXCR3+ T cells in tumors of mice treated as indicated.

FIG. 6A shows gene expression of TGFβ in tumors of mice treated as indicated.

FIG. 6B shows gene expression of Stat6 in tumors of mice treated as indicated.

FIG. 7A shows gene expression of IFN-γ in tumors of mice treated as indicated.

FIG. 7B shows gene expression of Tbet in tumors of mice treated as indicated.

FIG. 8 shows gene expression of Klrc2 in tumors of mice treated as indicated.

FIGS. 9A, 9B, and 9C show the effects of anti-PD-1 and nanatinostat treatment on cell proliferation.

FIG. 9A, shows isolated PBMC that were stimulated with CEFT peptide for 10 days. During this period, proliferation was monitored until the cells became exhausted using 3H-Thymidine.

FIG. 9B, shows the percent of proliferating CD8+ cells in the control and Entinostat-treated cells.

FIG. 9C, shows the effect of nanatinostat with and without aPD-1 therapy on the percent of proliferating CD8+ cells. The solid black line represents the CEFT control and the dotted line represents the anti-PD-1 treated control.

FIGS. 10A and 10B show the effects of anti-PD-1 and nanatinostat treatment on cell viability.

FIG. 10A, shows the percent of viable cells in the controls and Entinostat-treated cells.

FIG. 10B, shows the effect of nanatinostat with and without anti-PD-1 therapy on the percentage of viable cells. The solid black line represents the CEFT control and the dotted line represents the anti-PD-1 treated control.

FIGS. 11A and 11B show the effects of anti-PD-1 and nanatinostat treatment on IFN-γ release by CD8+ T cells.

FIG. 11A, shows the percent of IFNγ secreting CD8+ cells in the controls and Entinostat-treated cells.

FIG. 11B, shows the effect of nanatinostat with and without anti-PD-1 therapy on the percent of IFNγ secreting CD8+ cells. The solid black line represents the CEFT control and the dotted line represents the anti-PD-1 treated control.

FIGS. 12A, 12B and 12C show the effects of anti-PD-1 and nanatinostat treatment on IFN-γ, TNFα, and TGFβ. Isolated PBMC were exhausted with CEFT-stimulation for 10 days prior to being restimulated with moDC and CEFT peptide with compound treatment for an additional 4 days. Luminex analysis was performed and levels of IFN-γ (FIG. 12A), TNFα (FIG. 12B), and TGFβ (FIG. 12C). Dotted lines denote anti-PD-1 control treatment and vehicle control treatment as indicated.

### DETAILED DESCRIPTION OF THE INVENTION

In a certain aspect, described herein, is a method for augmenting a cell-based immunotherapy comprising contacting a cell-based immunotherapy in vitro with an HDAC inhibitor (HDACi), wherein the HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide).

In another aspect, described herein, is a method of adoptive cell immunotherapy comprising: a) contacting a cell-based immunotherapy with an HDAC inhibitor (HDACi), wherein the HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide); and b) administering the cell-based immunotherapy to an individual afflicted with a disease.

In another aspect, described herein, is a cell culture media comprising an HDAC inhibitor (HDACi), wherein the HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide).

Provided herein are methods for treating and/or preventing a disease in an individual in need thereof. In certain embodiments, the disease is a cancer. In certain embodiments, the treatment can comprise the steps of contacting a cell-based immunotherapy in vitro with an effective amount of an HDACi. In certain embodiments, the cell-based immunotherapy comprises a T cell (CD4+ or CD8+). In certain embodiments, the method further comprises administering the cell-based immunotherapy that has been contacted in vitro to a patient afflicted with a cancer. In certain embodiments, the HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide).

Also provided herein are compositions and cell culture media for treating and/or preventing a disease in an individual in need thereof. In certain embodiments, the disease is a cancer. In certain embodiments, the disease is associated with a cancer. In certain embodiments, the composition comprises an HDAC inhibitor suspended in a cell culture medium. In certain embodiments, the HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide). In certain embodiments, the cell culture medium comprises a cell-based immunotherapy.

In another aspect, described herein, is a method of treating human immunodeficiency (HIV) infection in an individual comprising: administering to an individual with an HIV infection an effective amount of nanatinostat, wherein the individual with an HIV infection has an HIV viral load of less than 1000 copies of HIV RNA per milliliter of blood.

In another aspect, described herein, is a method for treating an individual with a latent viral infection comprising: a) administering to an individual with the latent viral infection a first histone deactylase inhibitor (HDACi); b) contacting a cell-based immunotherapy in vitro with a second HDACi, wherein the second HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide); and c) administering the cell-based immunotherapy to individual with the latent viral infection.

In another aspect, described herein, is a method for treating an individual with a latent viral infection comprising: a) administering to an individual with the latent viral infection a first histone deactylase inhibitor (HDACi), wherein the first HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide)); b) contacting a cell-based immunotherapy in vitro with a second HDACi; and c) administering the cell-based immunotherapy to the individual with the latent viral infection.

The term “about,” as used herein, refers to a number within 10% of the stated amount.

The terms “comprises” and “comprising” are intended to have the broad meaning ascribed to them and can mean “includes,” “including,” and the like.

The term “subject,” “patient,” or “individual” are used interchangeably herein and refer to a human individual diagnosed with a disorder described herein, suffering from a disorder described herein, at risk of suffering from a disorder described herein, suspected of suffering from a disorder described herein, including individuals who may be asymptomatic or prodromal. In certain embodiments, individual refers to a donor or source of a cell-based therapeutic.

The terms “treat,” “treating,” or “treatment,” and other grammatical equivalents as used herein, include alleviating, inhibiting, or reducing symptoms, reducing or inhibiting severity of, reducing incidence of, prophylactic treatment of, reducing or inhibiting recurrence of, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease, to a patient reporting one or more of the physiological symptoms of a disease, or to a patient at risk of reoccurrence of the disease.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that are used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion). Administration techniques that in some instances are employed with the agents and methods described herein include, e.g., as discussed in Goodman and Gilman, The Pharmacological Basis of Therapeutics (current edition), Pergamon; and Remington's, Pharmaceutical Sciences (current edition), Mack Publishing Co., Easton, Pa. In certain embodiments, the agents and compositions described herein are administered orally. In some embodiments, the compositions described herein are administered parenterally.

In some embodiments, the compositions and methods herein will “consist essentially” of the recited steps or components. It is meant that consists essentially means that the recited steps or components contribute to the functional or therapeutic effect, and no other components or steps are included that contribute to the functional or therapeutic effect. A method that consists essentially can include steps that are not necessary to the functional or therapeutic effect on the cell-based immunotherapy; non-limiting examples include purification/isolation steps, cell expansion steps, cell maintenance steps, chemicals, chemicals added to reach a certain tonicity, vitamin supplements, pH buffers or modifiers, energy sources, fatty acids, sugars, polypeptides, proteins, growth factors, feeder cells that are added to maintain/expand cells in culture. A composition that consists essentially can include components that are not necessary to the functional or therapeutic effect on the cell-based immunotherapy; non-limiting examples include chemicals, chemicals added to reach a certain tonicity, vitamin supplements, pH buffers or modifiers, energy sources, fatty acids, sugars, polypeptides, proteins, growth factors, and feeder cells that are added to maintain/expand cells in culture.

### HDAC Inhibitors

The methods of the provided invention comprise use of one or more compositions or methods provided herein comprising an HDAC inhibitor (HDACi). The HDAC inhibitor is contacted with a cell-based immunotherapy to reverse the phenomena of exhaustion or to otherwise augment the therapy. The HDACi can be co-cultured with a cell-based immunotherapy, or alternatively the HDACi can be administered to an individual before isolation of lymphocytes, T cells or NK cells, from that individual. The subsequently isolated lymphocytes, T cells, or NK cells can be isolated from peripheral blood mononuclear cells (PBMCs), or from the tumor directly (tumor infiltrating lymphocytes).

For in vitro applications (e.g., administration in cell culture) a cell-based immunotherapy can be treated or contacted with an effective amount of the HDACi. An effective amount is one that results in increased histone acetylation. In a certain embodiment, the histone with increased acetylation comprises Histone H3. In a certain embodiment, the histone with increased acetylation comprises Histone H3, and the increased acetylation is at lysine 9. In certain embodiments, the cell-based immunotherapy is treated with a concentration of HDACi less than about 10 μM. In certain embodiments, the cell-based immunotherapy is treated with a concentration of HDACi less than about 5 μM. In certain embodiments, the cell-based immunotherapy is treated with a concentration of HDACi less than about 2 μM. In certain embodiments, the cell-based immunotherapy is treated with a concentration of HDACi less than about 1 μM. In certain embodiments, the cell-based immunotherapy is treated with a concentration of HDACi less than about 900 nM. In certain embodiments, the cell-based immunotherapy is treated with a concentration of HDACi less than about 800 nM. In certain embodiments, the cell-based immunotherapy is treated with a concentration of HDACi less than about 700 nM. In certain embodiments, the cell-based immunotherapy is treated with a concentration of HDACi less than about 600 nM. In certain embodiments, the cell-based immunotherapy is treated with a concentration of HDACi less than about 500 nM. In certain embodiments, the cell-based immunotherapy is treated with a concentration of HDACi less than about 400 nM. In certain embodiments, the cell-based immunotherapy is treated with a concentration of HDACi less than about 300 nM. In certain embodiments, the cell-based immunotherapy is treated with a concentration of HDACi less than about 200 nM. In certain embodiments, the cell-based immunotherapy is treated with a concentration of HDACi less than about 100 nM. In certain embodiments, the cell-based immunotherapy is treated with a concentration of HDACi less than about 50 nM. In certain embodiments, the cell-based immunotherapy is treated with a concentration of HDACi greater than about 1 nM. In certain embodiments, the cell-based immunotherapy is treated with a concentration of HDACi greater than about 2 nM. In certain embodiments, the cell-based immunotherapy is treated with a concentration of HDACi greater than about 5 nM. In certain embodiments, the cell-based immunotherapy is treated with a concentration of HDACi greater than about 10 nM. In certain embodiments, the cell-based immunotherapy is treated with a concentration of HDACi greater than about 100 nM. In certain embodiments, the HDACi is administered between about 1 nM and about 5 μM, between about 1 nM and about 2 μM, between about 1 nM and about 1 μM, between about 1 nM and about 900 nM, between about 1 nM and about 800 nM, between about 1 nM and about 700 nM, between about 1 nM and about 600 nM, between about 1 nM and about 500 nM, between about 1 nM and about 400 nM, between about 1 nM and about 300 nM, between about 1 nM and about 200 nM, between about 1 nM and about 100 nM, between about 1 nM and about 50 nM, between about 1 nM and about 25 nM, between about 10 nM and about 5 μM, between about 10 nM and about 2 μM, between about 10 nM and about 1 μM, between about 10 nM and about 900 nM, between about 10 nM and about 800 nM, between about 10 nM and about 700 nM, between about 10 nM and about 600 nM, between about 10 nM and about 500 nM, between about 10 nM and about 400 nM, between about 10 nM and about 300 nM, between about 10 nM and about 200 nM, between about 1 nM and about 100 nM, between about 10 nM and about 50 nM, between about 10 nM and about 25 nM. The HDACi can be incubated with a cell-based immunotherapy for about 1, 2, 4, 8, 16, 24, or 48 hours. The HDACi can be incubated with a cell-based immunotherapy for at least about 1, 2, 4, 8, 16, 24, or 48 hours. The HDACi can be incubated with a cell-based immunotherapy for no more than about 1, 2, 4, 8, 16, 24, or 48 hours.

In certain embodiments, the HDACi comprises a histone deacetylase complex inhibitor (HDACi), wherein the HDACi comprises quisinostat (JNJ-26481585 (N-hydroxy-2-(4-((((1-methyl-1H-indol-3-yl)methyl)amino)methyl)piperidin-1-yl)pyrimidine-5-carboxamide)), R306465/JNJ-16241199 (N-hydroxy-5-(4-(naphthalen-2-ylsulfonyl)piperazin-1-yl)pyrimidine-2-carboxamide), Belinostat/PXD101, trichostatin A/TSA (7-[4-(dimethylamino)phenyl]-N-hydroxy-4,6-dimethyl-7-oxohepta-2,4-dienamide), ITF2357, CBHA, Givinostat/ITF2357, romidepsin, PCI-24781, depsipeptide (FR901228 or FK228), butyrate, phenylbutyrate, valproic acid, AN-9, CI-994, Entinostat/MS-275/SNDX-275, mocetinostat/MGCD0103 (N-(2-aminophenyl)-4-((4-pyridin-3-ylpyrimidin-2-ylamino)methyl)benzamide), m-carboxycinnamic acid, bishydroxamic acid, suberic bishydroxamic acid, oxamflatin, ABHA, SB-55629, pyroxamide, propenamides, aroyl pyrrolyl hydroxamides, or LAQ824 (((E)-N-hydroxy-3-[4-[[2-hydroxyethyl-[2-(1H-indol-3-yl)ethyl]amino]methyl]phenyl]prop-2-enamide), panobinostat/LBH-589, vorinsotat/SAHA, chidamide, or 4SC-202.

For in vivo applications a patient can be treated with an effective amount of the HDACi before cells are isolated from the patient. In certain embodiments, the HDAC inhibitor is administered at a dose of less than 400 mg/day. In some embodiments, the HDAC inhibitor is administered at a dose of about 1 mg/day, about 2 mg/day, about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 60 mg/day, about 70 mg/day, about 80 mg/day, about 90 mg/day, about 100 mg/day, about 120 mg/day, about 125 mg/day, about 140 mg/day, about 150 mg/day, about 160 mg/day, about 175 mg/day, about 180 mg/day, about 190 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, or about 500 mg/day. In certain embodiments, the HDAC inhibitor is administered at a dose of less than 1 mg/day, less than 2 mg/day, less than 5 mg/day, less than 10 mg/day, less than 15 mg/day, less than 20 mg/day, less than 25 mg/day, less than 30 mg/day, less than 35 mg/day, less than 40 mg/day, less than 45 mg/day, less than 50 mg/day, less than 60 mg/day, less than 70 mg/day, less than 80 mg/day, less than 90 mg/day, less than 100 mg/day, less than 120 mg/day, less than 125 mg/day, less than 140 mg/day, less than 150 mg/day, less than 160 mg/day, less than 175 mg/day, less than 180 mg/day, less than 190 mg/day, less than 200 mg/day, less than 225 mg/day, less than 250 mg/day, less than 275 mg/day, less than 300 mg/day, less than 325 mg/day, less than 350 mg/day, less than 375 mg/day, less than 400 mg/day, less than 425 mg/day, less than 450 mg/day, less than 475 mg/day, or less than 500 mg/day. In some embodiments, the HDAC inhibitor is administered at a dose of more than 1 mg/day, more than 2 mg/day, more than 5 mg/day, more than 10 mg/day, more than 15 mg/day, more than 20 mg/day, more than 25 mg/day, more than 30 mg/day, more than 35 mg/day, more than 40 mg/day, more than 45 mg/day, more than 50 mg/day, more than 60 mg/day, more than 70 mg/day, more than 80 mg/day, more than 90 mg/day, more than 100 mg/day, more than 120 mg/day, more than 125 mg/day, more than 140 mg/day, more than 150 mg/day, more than 160 mg/day, more than 175 mg/day, more than 180 mg/day, more than 190 mg/day, more than 200 mg/day, more than 225 mg/day, more than 250 mg/day, more than 275 mg/day, more than 300 mg/day, more than 325 mg/day, more than 350 mg/day, more than 375 mg/day, more than 400 mg/day, more than 425 mg/day, more than 450 mg/day, more than 475 mg/day, or more than 500 mg/day. In certain embodiments, the HDAC inhibitor is administered at a dose of more than 1 mg/day and less than 500 mg/day. In some embodiments, the HDAC inhibitor is administered at a dose of more than 20 mg/day and less than 80 mg/day. In certain embodiments, the HDAC inhibitor is administered once a day (q.d.), twice a day (b.i.d.), or thrice a day (t.i.d.). In some embodiments, the HDAC inhibitor is administered daily, once a week, twice a week, three times a week, four times a week, or five times a week.

In certain embodiments, the HDACi comprises a histone deacetylase complex inhibitor (HDACi), wherein the HDACi comprises quisinostat (JNJ-26481585 (N-hydroxy-2-(4-((((1-methyl-1H-indol-3-yl)methyl)amino)methyl)piperidin-1-yl)pyrimidine-5-carboxamide)), R306465/JNJ-16241199 (N-hydroxy-5-(4-(naphthalen-2-ylsulfonyl)piperazin-1-yl)pyrimidine-2-carboxamide), Belinostat/PXD101, trichostatin A/TSA (7-[4-(dimethylamino)phenyl]-N-hydroxy-4,6-dimethyl-7-oxohepta-2,4-dienamide), ITF2357, CBHA, Givinostat/ITF2357, romidepsin, PCI-24781, depsipeptide (FR901228 or FK228), butyrate, phenylbutyrate, valproic acid, AN-9, CI-994, Entinostat/MS-275/SNDX-275, mocetinostat/MGCD0103 (N-(2-aminophenyl)-4-((4-pyridin-3-ylpyrimidin-2-ylamino)methyl)benzamide), m-carboxycinnamic acid, bishydroxamic acid, suberic bishydroxamic acid, oxamflatin, ABHA, SB-55629, pyroxamide, propenamides, aroyl pyrrolyl hydroxamides, or LAQ824 (((E)-N-hydroxy-3-[4-[[2-hydroxyethyl-[2-(1H-indol-3-yl)ethyl]amino]methyl]phenyl]prop-2-enamide), panobinostat/LBH-589, vorinsotat/SAHA, chidamide, or 4SC-202.

For in vitro applications (e.g., administration in cell culture) a cell-based immunotherapy can be treated or contacted with an effective amount of a class I HDACi. In some embodiments, the class I HDACi is Nanatinostat (also referred to as Nstat, tractinostat, VRx-3996, or CHR-3996). The chemical formula of Nanatinostat is (2-(6-{[(6-Fluoroquinolin-2-yl)methyl]amino}-3-azabicyclo[3.1.0]hex-3-yl)-N-hydroxypyrimidine-5-carboxamide). Nanatinostat is a selective Class I HDAC inhibitor and is disclosed in U.S. Pat. No. 7,932,246, which is incorporated by reference herein in its entirety. An effective amount is one that results in increased histone acetylation in a cell-based immunotherapeutic. In a certain embodiment, the histone with increased acetylation comprises Histone H3. In a certain embodiment, the histone with increased acetylation comprises Histone H3 and the increased acetylation is at lysine 9. In certain embodiments, the cell-based immunotherapy is treated with a concentration of nanatinostat less than about 10 μM. In certain embodiments, the cell-based immunotherapy is treated with a concentration of nanatinostat less than about 5 μM. In certain embodiments, the cell-based immunotherapy is treated with a concentration of nanatinostat less than about 2 μM. In certain embodiments, the cell-based immunotherapy is treated with a concentration of nanatinostat less than about 1 μM. In certain embodiments, the cell-based immunotherapy is treated with a concentration of nanatinostat less than about 900 nM. In certain embodiments, the cell-based immunotherapy is treated with a concentration of nanatinostat less than about 800 nM. In certain embodiments, the cell-based immunotherapy is treated with a concentration of nanatinostat less than about 700 nM. In certain embodiments, the cell-based immunotherapy is treated with a concentration of nanatinostat less than about 600 nM. In certain embodiments, the cell-based immunotherapy is treated with a concentration of nanatinostat less than about 500 nM. In certain embodiments, the cell-based immunotherapy is treated with a concentration of nanatinostat less than about 400 nM. In certain embodiments, the cell-based immunotherapy is treated with a concentration of nanatinostat less than about 300 nM. In certain embodiments, the cell-based immunotherapy is treated with a concentration of nanatinostat less than about 200 nM. In certain embodiments, the cell-based immunotherapy is treated with a concentration of nanatinostat less than about 100 nM. In certain embodiments, the cell-based immunotherapy is treated with a concentration of nanatinostat less than about 50 nM. In certain embodiments, the cell-based immunotherapy is treated with a concentration of nanatinostat greater than about 1 nM. In certain embodiments, the cell-based immunotherapy is treated with a concentration of nanatinostat greater than about 2 nM. In certain embodiments, the cell-based immunotherapy is treated with a concentration of nanatinostat greater than about 5 nM. In certain embodiments, the cell-based immunotherapy is treated with a concentration of nanatinostat greater than about 10 nM. In certain embodiments, the cell-based immunotherapy is treated with a concentration of nanatinostat greater than about 100 nM. In certain embodiments, the nanatinostat is administered between about 1 nM and about 5 μM, between about 1 nM and about 2 between about 1 nM and about 1 μM, between about 1 nM and about 900 nM, between about 1 nM and about 800 nM, between about 1 nM and about 700 nM, between about 1 nM and about 600 nM, between about 1 nM and about 500 nM, between about 1 nM and about 400 nM, between about 1 nM and about 300 nM, between about 1 nM and about 200 nM, between about 1 nM and about 100 nM, between about 1 nM and about 50 nM, between about 1 nM and about 25 nM, between about 10 nM and about 5 between about 10 nM and about 2 between about 10 nM and about 1 μM, between about 10 nM and about 900 nM, between about 10 nM and about 800 nM, between about 10 nM and about 700 nM, between about 10 nM and about 600 nM, between about 10 nM and about 500 nM, between about 10 nM and about 400 nM, between about 10 nM and about 300 nM, between about 10 nM and about 200 nM, between about 1 nM and about 100 nM, between about 10 nM and about 50 nM, between about 10 nM and about 25 nM. In certain embodiments, the cell-based immunotherapy is treated with a concentration of nanatinostat that is about 1 μM. In certain embodiments, the cell-based immunotherapy is treated with a concentration of nanatinostat that is about 900 nM. In certain embodiments, the cell-based immunotherapy is treated with a concentration of nanatinostat that is about 800 nM. In certain embodiments, the cell-based immunotherapy is treated with a concentration of nanatinostat that is about 700 nM. In certain embodiments, the cell-based immunotherapy is treated with a concentration of nanatinostat that is about 600 nM. In certain embodiments, the cell-based immunotherapy is treated with a concentration of nanatinostat that is about 500 nM. In certain embodiments, the cell-based immunotherapy is treated with a concentration of nanatinostat that is about 400 nM. In certain embodiments, the cell-based immunotherapy is treated with a concentration of nanatinostat that is about 300 nM. In certain embodiments, the cell-based immunotherapy is treated with a concentration of nanatinostat that is about 200 nM. In certain embodiments, the cell-based immunotherapy is treated with a concentration of nanatinostat that is about 100 nM.

In certain embodiments, the cell-based immunotherapy is treated with a concentration of nanatinostat from about 100 nM to about 1,000 nM. In certain embodiments, the cell-based immunotherapy is treated with a concentration of nanatinostat from about 100 nM to about 200 nM, about 100 nM to about 300 nM, about 100 nM to about 400 nM, about 100 nM to about 500 nM, about 100 nM to about 600 nM, about 100 nM to about 700 nM, about 100 nM to about 800 nM, about 100 nM to about 900 nM, about 100 nM to about 1,000 nM, about 200 nM to about 300 nM, about 200 nM to about 400 nM, about 200 nM to about 500 nM, about 200 nM to about 600 nM, about 200 nM to about 700 nM, about 200 nM to about 800 nM, about 200 nM to about 900 nM, about 200 nM to about 1,000 nM, about 300 nM to about 400 nM, about 300 nM to about 500 nM, about 300 nM to about 600 nM, about 300 nM to about 700 nM, about 300 nM to about 800 nM, about 300 nM to about 900 nM, about 300 nM to about 1,000 nM, about 400 nM to about 500 nM, about 400 nM to about 600 nM, about 400 nM to about 700 nM, about 400 nM to about 800 nM, about 400 nM to about 900 nM, about 400 nM to about 1,000 nM, about 500 nM to about 600 nM, about 500 nM to about 700 nM, about 500 nM to about 800 nM, about 500 nM to about 900 nM, about 500 nM to about 1,000 nM, about 600 nM to about 700 nM, about 600 nM to about 800 nM, about 600 nM to about 900 nM, about 600 nM to about 1,000 nM, about 700 nM to about 800 nM, about 700 nM to about 900 nM, about 700 nM to about 1,000 nM, about 800 nM to about 900 nM, about 800 nM to about 1,000 nM, or about 900 nM to about 1,000 nM. In certain embodiments, the cell-based immunotherapy is treated with a concentration of nanatinostat at about 100 nM, about 200 nM, about 300 nM, about 400 nM, about 500 nM, about 600 nM, about 700 nM, about 800 nM, about 900 nM, or about 1,000 nM. In certain embodiments, the cell-based immunotherapy is treated with a concentration of nanatinostat from at least about 100 nM, about 200 nM, about 300 nM, about 400 nM, about 500 nM, about 600 nM, about 700 nM, about 800 nM, or about 900 nM. In certain embodiments, the cell-based immunotherapy is treated with a concentration of nanatinostat of no more than about 200 nM, about 300 nM, about 400 nM, about 500 nM, about 600 nM, about 700 nM, about 800 nM, about 900 nM, or about 1,000 nM.

Nanatinostat can be incubated with a cell-based immunotherapy for about 1, 2, 4, 8, 16, 24, or 48 hours. Nanatinostat can be incubated with a cell-based immunotherapy for at least about 1, 2, 4, 8, 16, 24, or 48 hours. Nanatinostat can be incubated with a cell-based immunotherapy for no more than about 1, 2, 4, 8, 16, 24, or 48 hours.

For in vivo applications a patient can be treated with an effective amount of a class I HDAC inhibitor. In some embodiments, the class I HDACi is nanatinostat. In certain embodiments, nanatinostat administered at a dose of 40 mg/day. In some embodiments, Nanatinostat is administered at a dose of about 1 mg/day, about 2 mg/day, about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 60 mg/day, about 70 mg/day, about 80 mg/day, about 90 mg/day, or about 100 mg/day. In certain embodiments, Nanatinostat is administered at a dose of less than 1 mg/day, less than 2 mg/day, less than 5 mg/day, less than 10 mg/day, less than 15 mg/day, less than 20 mg/day, less than 25 mg/day, less than 30 mg/day, less than 35 mg/day, less than 40 mg/day, less than 45 mg/day, less than 50 mg/day, less than 60 mg/day, less than 70 mg/day, less than 80 mg/day, less than 90 mg/day, or less than 100 mg/day. In some embodiments, Nanatinostat is administered at a dose of more than 1 mg/day, more than 2 mg/day, more than 5 mg/day, more than 10 mg/day, more than 15 mg/day, more than 20 mg/day, more than 25 mg/day, more than 30 mg/day, more than 35 mg/day, more than 40 mg/day, more than 45 mg/day, more than 50 mg/day, more than 60 mg/day, more than 70 mg/day, more than 80 mg/day, more than 90 mg/day, or more than 100 mg/day. In certain embodiments, nanatinostat is administered at a dose of more than 30 mg/day and less than 50 mg/day. In some embodiments, nanatinostat is administered at a dose of more than 5 mg/day and less than 80 mg/day. In some embodiments, nanatinostat is administered at a dose of more than 10 mg/day and less than 80 mg/day. In some embodiments, nanatinostat is administered at a dose of more than 20 mg/day and less than 80 mg/day. In some embodiments, nanatinostat is administered at a dose of about 1 mg/day, about 2 mg/day, about 5 mg/day, about 6 mg/day, about 7 mg/day, about 8 mg/day, about 9 mg/day, about 10 mg/day, about 11 mg/day, about 12 mg/day, about 13 mg/day, about 14 mg/day, about 15 mg/day, about 16 mg/day, about 17 mg/day, about 18 mg/day, about 19 mg/day, about 20 mg/day, about 22 mg/day, about 23 mg/day, about 25 mg/day, about 27 mg/day, about 28 mg/day, about 30 mg/day, about 32 mg/day, about 33 mg/day, about 35 mg/day, about 40 mg/day, about 45 mg/day, about 50 mg/day, about 60 mg/day, about 70 mg/day, about 80 mg/day, about 90 mg/day, or about 100 mg/day. In certain embodiments, nanatinostat is administered once a day (q.d.), twice a day (b.i.d.), or thrice a day (t.i.d.). In some embodiments, nanatinostat is administered daily, once a week, twice a week, three times a week, four times a week, or five times a week.

### Cell-Based Immunotherapies

The HDACi described herein are for use in a method of augmenting a cell-based therapy. The HDACi can be applied in vitro to cell-based immunotherapies in culture. These cell-based immunotherapies can be manufactured from a cell population isolated from a patient to be treated or an HLA matched donor. The HDACi can be used to treat a cell line or a primary cell population from a non-HLA matched donor. Alternatively, the HDACi can be used to treat a patient or healthy donor before isolation of a cell population to be used in manufacturing a cell-based immunotherapy.

In certain embodiments, the method(s) described herein are methods of augmenting T cell based immunotherapies. In certain embodiments, the method described herein is a method of increasing IFN-γ expression or secretion in a cell-based immunotherapy. In certain embodiments, the method described herein is a method of increasing TNFα expression or secretion in a cell-based immunotherapy. In certain embodiments, the method described herein is a method of reducing TGFβ expression or secretion in a cell-based immunotherapy.

Cell-based immunotherapies generally comprise immune effector cells such as T cells, and NK cells, and antigen presenting cells (e.g., macrophages, dendritic cells, and B cells). The HDACi disclosed herein are useful for augmenting these cell-based immunotherapies. The cell-based immunotherapy can be one or more adoptively transferred lymphocyte populations that comprise T cells, a T-cell population, or a T cell line. Alternatively, the cell-based immunotherapy can be NK cells, an NK-cell population or an NK cell line. In certain embodiments, the cell based immunotherapy that is augmented is a population of cells that is antigen experienced, and has been rendered functionally anergic, functionally deficient, or exhausted. Exhaustion (or functional deficiency) can be evidenced in T cells by reduced levels of cytotoxicity against a target cell population, trafficking to a tumor/infection site, IFN-γ expression/secretion, CXCR3 expression, or T-bet. Functional deficiency in T cells or a T-cell response can also be evidenced by high levels of regulatory T cells (T_{REG}) marked by FoxP3 transcription factor expression. Exhaustion (or functional deficiency) can be evidenced in NK cells by reduced levels of cytotoxicity against a target cell population expression secretion of IFN-γ or GMCSF, perforin, or granzyme B; or reduced expression of FasL or TRAIL. In certain embodiments, the cell-based immunotherapy can be a therapeutic vaccine.

### T Cell Based Therapies

In certain embodiments, the cell-based immunotherapy to be treated with an HDACi herein is a population of lymphocytes. In certain embodiments, the population of lymphocytes is derived from peripheral blood mononuclear cells (PBMCs) isolated from the circulation of an individual. In certain embodiments, the population of lymphocytes is derived from lymphocytes isolated from a tumor (tumor infiltrating lymphocytes) of an individual. In certain embodiments, the population of lymphocytes comprises T lymphocytes (T cells). These cell populations can be heterogeneous comprised of a variety of lymphocytes, or they can be further subject to isolation/purification using density centrifugation (e.g., Percoll), fluorescently activated cell sorting (FACS), leukapheresis, or antibody based selection methods (positive or negative). T cells can be generally marked by expression of CD3, and further subdivided into cytotoxic (CD8+) or helper (CD4+) populations. When isolated/purified the cell population can comprise CD3+ cells at least 80%, 90%, or 95% pure. In certain embodiments, the population comprises CD3+, CD4+ T cells at least 80%, 90%, or 95% pure. In certain embodiments, the population comprises CD3+, CD8+ T cells at least 80%, 90%, or 95% pure. T-cell populations can be further isolated and selected for low expression of checkpoint inhibitors such as CTLA4, LAG-3 or PD-1.

Isolated and purified cell populations can be further expanded using standard methods, such as, incubation with anti-CD3 or CD28 antibody and/or co-culture with cytokines such as IL-2, IL-7 and/or IL-15. In certain embodiments, the isolated and purified cell population is incubated with irradiated feeder cells and peptide antigen to expand one or more T cells of a certain antigen specificity. In certain embodiments, the peptide antigen comprises a tumor associated antigen.

Heterogeneous cell populations can be further expanded using standard methods such as incubation with anti-CD3 or CD28 antibody and/or co-culture with cytokines such as IL-2, IL-7 and/or IL-15. In certain embodiments, the isolated and purified cell population is incubated with irradiated feeder cells and peptide antigen to expand one or more T cells of a certain antigen specificity. In certain embodiments, the peptide antigen comprises a tumor associated antigen. After the cells have been expanded the cells can comprise greater than 60%, 70%, 80%, 90%, or 95% CD3+ cells, CD3+CD4+ cells, or CD3+CD8+ cells. In certain embodiments, an aliquot of the cells can be tested for efficacy after expansion.

There are numerous methods available for isolating or expanding T cells or T-cell populations taken from an individual. Certain non-limiting methods of expanding and/or isolating T-cell populations are disclosed in U.S. Pat. Nos. 5,827,642; 6,316,257; 6,399,054; 7,745,140; 8,383,099; US 2003/0134341; US 2004/0241162; all of which are incorporated by reference herein in their entireties.

T cell populations can also be derived from hematopoietic stem cells (HSCs) or induced pluripotent stem cells (iPSCs) using methods known in the art. In certain embodiments, T-cell populations are derived/differentiated from iPSCs. The source of the iPSCs can be either autologous or heterologous. In certain embodiments, T-cell populations are derived/differentiated from (HSCs) cells. The source of the HSCs can be either autologous or heterologous.

T-cell populations to be treated by the HDACi herein can be derived from an individual that will ultimately be treated with the cell-based immunotherapeutic (e.g., an autologous population) or from a different individual (e.g., a heterologous population). In certain embodiments, when an autologous cell population is used the cell population has been treated in vitro with an HDACi. In certain embodiments, when an autologous cell population is used that person has been administered an HDACi on one or more occasions prior to isolation of the cell population. In certain embodiments, when a heterologous cell population is used it is from an HLA matched individual (e.g., syngeneic) or an HLA mismatched individual (e.g., allogeneic). In certain embodiments, when a heterologous cell population is used it is from an HLA mismatched donor. In certain embodiments, when a heterologous cell population is used it is a T cell line that can be established from an autologous or heterologous source.

When a T-cell population (either heterogeneous or purified; autologous or heterologous) or a T-cell line is utilized in the methods described herein, the population can be stimulated or activated by a specific tumor-associated antigen either before or after treatment with an HDACi. A tumor associated antigen (TAA) is one that is exclusively expressed or highly expressed by a neoplastic cell compared to a normal cell of the same origin. Known tumor-associated antigens include, for example, glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, MART-1, Lewis Y antigen (LeY), tyrosinase and GP 100, prostatic acid phosphatase (PAP) prostate-specific antigen (PSA), ROR1, MUC16, CD171 (LICAM), B-cell maturation antigen (BCMA), WT1, HER-2/Neu/ErbB-2, CD19, CD20, CD37, or patient specific idiotype. In certain embodiments, greater than 50%, 60%, 70%, 80%, 90%, or 95% of the T-cell population can be specific for a tumor associated antigen (as defined by tetramer staining for example). In certain embodiments, the T-cell population may not be stimulated with TAA, but may possess specificity for the TAA, as indicated for example, by tetramer staining. In certain embodiments, the T-cell population may not be stimulated with viral antigen, but may possess specificity for the viral antigen, as indicated for example, by tetramer staining.

When a T-cell population (either heterogeneous or purified; autologous or heterologous) or a T-cell line is utilized in the methods described herein, the population can be stimulated or activated by a viral antigen derived from human cytomegalovirus, Epstein-Barr virus, human immunodeficiency virus, hepatitis C virus, or hepatitis B virus. In certain embodiments, the population is stimulated by an antigen derived from Epstein-Barr virus. In certain embodiments, the population is stimulated by an antigen derived from human cytomegalovirus.

Immune responses are negatively regulated by CD4+ T regulatory cells. Reduction of CD4+ Tregs is an important strategy for increasing therapeutic responses to cell-based immune therapies. FoxP3 is a transcriptional regulator of regulatory T cell phenotypes. In certain embodiments, the HDAC inhibitors described herein reduce FoxP3+, CD4+ T regulatory cell populations in vitro. In certain embodiments, the HDAC inhibitors described herein reduce FoxP3+, CD4+ T regulatory cell populations by at least 10%, 20%, 30%, 40%, 50, 60%, 70% or more. These T-cell populations can be reduced in an induvial after dosing with an HDAC inhibitor but prior to isolation of the cells for use in a cell-based immunotherapy. In certain embodiments, the HDAC inhibitors described herein reduce FoxP3+, CD4+ T regulatory cell populations by at least 10%, 20%, 30%, 40%, 50, 60%, 70%, 80%, 90%, 95% or more in an induvial treated with HDAC inhibitor compared to a placebo treated individual. In certain embodiments, the HDAC inhibitors described herein reduce FoxP3+, CD4+ T regulatory cell populations by at least 10%, 20%, 30%, 40%, 50, 60%, 70% or more in ex vivo cultured peripheral blood mononuclear cells compared to PBMC treated with a vehicle control or left untreated.

T-cell populations and T-cell lines used in the method described herein display augmented functionality. This functionality can be a physiological function such as increased half-life in the circulation, higher trafficking to tumor sites, increased cytotoxic activity, or increased cytokine/chemokine secretion compared to a non-HDACi treated T-cell population. In certain embodiments, the HDACi treated cell population or cell line exhibits a half-life that is 10%, 25%, 50%, or 75% longer than a non-HDACi treated cell population or cell line. In certain embodiments, the HDACi treated cell population or cell line exhibits a half-life that is 2-fold, 3-fold, 4-fold, or 5-fold longer than a non-HDACi treated cell population or cell line. In certain embodiments, the HDACi treated cell population or cell line exhibits trafficking to a tumor site that is 10%, 25%, 50%, or 75% greater than a non-HDACi treated cell population or cell line. In certain embodiments, the HDACi treated cell population or cell line exhibits trafficking to a tumor site that is 2-fold, 3-fold, 4-fold, or 5-fold greater than a non-HDACi treated cell population or cell line. In certain embodiments, the HDACi treated cell population or cell line exhibits cytotoxic activity that is 10%, 25%, 50%, or 75% greater than a non-HDACi treated cell population or cell line. In certain embodiments, the HDACi treated cell population or cell line exhibits cytotoxic activity that is 2-fold, 3-fold, 4-fold, or 5-fold greater than a non-HDACi treated cell population or cell line. In certain embodiments, the HDACi treated cell population or cell line releases IFN-γ at a level 10%, 25%, 50%, or 75% greater than a non-HDACi treated cell population or cell line. In certain embodiments, the HDACi treated cell population or cell line releases IFN-γ at a level 2-fold, 3-fold, 4-fold, or 5-fold greater than a non-HDACi treated cell population or cell line. In certain embodiments, the HDACi treated cell population or cell line releases IL-2 at a level 10%, 25%, 50%, or 75% greater than a non-HDACi treated cell population or cell line. In certain embodiments, the HDACi treated cell population or cell line releases IL-2 at a level 2-fold, 3-fold, 4-fold, or 5-fold greater than a non-HDACi treated cell population or cell line. A non-HDACi treated cell population or cell line can for example, be a comparison of a before and after treatment or comparison to a similarly treated cell population except for HDACi treatment.

Alternatively, the increased functionality seen in a T-cell population or T-cell line can be a cellular or molecular function, such as increased expression of an activated cell-marker, reduced expression of an inhibitory cell-marker, increased cell-surface expression of an activated cell-marker, or reduced expression of an inhibitory cell-marker compared to a non-HDACi treated T-cell population. CXCR3 is a chemokine receptor that is preferentially expressed on activated Th₁ cells. In certain embodiments, the HDACi treated cell population or cell line expresses CXCR3 at the cell-surface at a level that is 10%, 25%, 50%, or 75% greater than a non-HDACi treated cell population or cell line. In certain embodiments, the HDACi treated cell population or cell line expresses CXCR3 at the cell-surface at a level that is 2-fold, 3-fold, 4-fold, or 5-fold greater than a non-HDACi treated cell population or cell line. FoxP3 is a transcription factor that is associated with T regulatory cells (T_{REG}). In certain embodiments, the HDACi treated cell population or cell line expresses FoxP3 at a level that is 10%, 25%, 50%, or 75% less than a non-HDACi treated cell population or cell line. In certain embodiments, the HDACi treated cell population or cell line expresses FoxP3 at a level that is 2-fold, 3-fold, 4-fold, or 5-fold less than a non-HDACi treated cell population or cell line. In certain embodiments, the HDACi treated cell population or cell line expresses IFN-γ mRNA at a level 10%, 25%, 50%, or 75% greater than a non-HDACi treated cell population or cell line. In certain embodiments, the HDACi treated cell population or cell line expresses IFN-γ mRNA at a level 2-fold, 3-fold, 4-fold, 5-fold, or 10-fold greater than a non-HDACi treated cell population or cell line. In certain embodiments, the HDACi treated cell population or cell line expresses TNFα mRNA at a level 10%, 25%, 50%, or 75% greater than a non-HDACi treated cell population or cell line. In certain embodiments, the HDACi treated cell population or cell line expresses TNFα mRNA at a level 2-fold, 3-fold, 4-fold, 5-fold, or 10-fold greater than a non-HDACi treated cell population or cell line. In certain embodiments, the HDACi treated cell population or cell line expresses IL-2 mRNA at a level 10%, 25%, 50%, or 75% greater than a non-HDACi treated cell population or cell line. In certain embodiments, the HDACi treated cell population or cell line expresses IL-2 mRNA at a level 2-fold, 3-fold, 4-fold, 5-fold, or 10-fold greater than a non-HDACi treated cell population or cell line. In certain embodiments, the HDACi treated cell population or cell line expresses T-bet mRNA at a level 10%, 25%, 50%, or 75% greater than a non-HDACi treated cell population or cell line. In certain embodiments, the HDACi treated cell population or cell line expresses T-bet mRNA at a level 2-fold, 3-fold, 4-fold, 5-fold, or 10-fold greater than a non-HDACi treated cell population or cell line. A non-HDACi treated cell population or cell line can for example, be a comparison of a before and after treatment or comparison to a similarly treated cell population except for HDACi treatment.

One way HDACi can augment T cell activity is by increases in or differentiation of T-cell populations into memory T cells. Memory T cells are highly active against targets expressing or displaying cognate antigen. In certain embodiments, the HDACi treated cell population or cell line expresses CCR7 at a level 10%, 25%, 50%, or 75% greater than a non-HDACi treated cell population or cell line. In certain embodiments, the HDACi treated cell population or cell line expresses CCR7 at a level 2-fold, 3-fold, 4-fold, 5-fold, or 10-fold greater than a non-HDACi treated cell population or cell line. In certain embodiments, the HDACi treated cell population or cell line expresses CD62L at a level 10%, 25%, 50%, or 75% greater than a non-HDACi treated cell population or cell line. In certain embodiments, the HDACi treated cell population or cell line expresses CD62L at a level 2-fold, 3-fold, 4-fold, 5-fold, or 10-fold greater than a non-HDACi treated cell population or cell line. In certain embodiments, the HDACi treated cell population or cell line expresses TGFβ at a level 10%, 25%, 50%, or 75% less than a non-HDACi treated cell population or cell line. In certain embodiments, the HDACi treated cell population or cell line expresses TGFβ at a level 2-fold, 3-fold, 4-fold, 5-fold, or 10-fold less than a non-HDACi treated cell population or cell line. A non-HDACi treated cell population or cell line can for example, be a comparison of a before and after treatment or comparison to a similarly treated cell population except for HDACi treatment.

T cells are additionally applied as cell-based therapeutics in conjunction with a chimeric antigen receptor (CAR), so called “CAR T cells.” CAR T cells are T cell lines or populations that have been genetically engineered to express a targeting domain (e.g., an antibody Fab or single chain variable fragment) fused to a transmembrane domain, and an intracellular domain that induces activation of the T cell upon interaction of the targeting domain with its target (e.g., CD3 zeta signaling domain, CD28 intracellular domain, 4-1BB intracellular domain). T cells can be made transgenic by viral transduction of a nucleic acid CAR construct into a primary T-cell population, using for example a retroviral, adenoviral, or AAV-vector; or transfection via a lipid-based reagent or electroporation. In certain embodiments, the methods described herein involve rendering a T-cell population transgenic before treatment with HDACi. In certain embodiments, the methods described herein involve rendering a T-cell population transgenic after treatment with HDACi. When CAR T cells are generated from a primary lymphocyte population the cells are often autologous to the patient being treated. The cells are isolated and expanded in culture using a conventional method such as CD3/CD28 antibodies to generate sufficient cells for the transduction and subsequent administration. Additionally, stable cell lines can also be established using CAR and administered. Current FDA approved CAR T cell therapies include axicabtagene ciloleucel (Yescarta™) and tisagenlecleucel (Kymriah™) CAR constructs and methods of their use are described in, by way of non-limiting example US20130287748A1; US 2014/0234348A1; or US 2014/0050708, all of which are incorporated by reference herein in their entirety.

In certain embodiments, the cell-based therapeutic is a T cell line or T-cell population rendered transgenic with a CAR. The population of T cells rendered transgenic with a CAR can express a targeting domain specific for a TAA, for example, glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, MART-1, Lewis Y antigen (LeY), tyrosinase and GP 100, prostatic acid phosphatase (PAP) prostate-specific antigen (PSA), ROR1, MUC16, CD171 (LICAM), B-cell maturation antigen (BCMA), WT1, HER-2/Neu/ErbB-2, CD19, CD20, or CD37.

When a T-cell line or T-cell population (either heterogeneous or purified; autologous or heterologous) is rendered transgenic by a CAR, the CAR can be specific for a viral antigen derived from human cytomegalovirus, Epstein-Barr virus, human immunodeficiency virus, hepatitis C virus, or hepatitis B virus. In certain embodiments, the population is stimulated by an antigen derived from Epstein-Barr virus. In certain embodiments, the population is stimulated by an antigen derived from human cytomegalovirus.

In certain embodiments, the CAR T cells are administered by i.v. infusion. In certain embodiments, about 1×10⁵ cells/m² are administered. In certain embodiments, about 2×10⁵ cells/m² are administered. In certain embodiments, about 3×10⁵ cells/m² are administered. In certain embodiments, about 4×10⁵ cells/m² are administered. In certain embodiments, about 5×10⁵ cells/m² are administered. In certain embodiments, about 6×10⁵ cells/m² are administered. In certain embodiments, about 7×10⁵ cells/m² are administered. In certain embodiments, about 8×10⁵ cells/m² are administered. In certain embodiments, about 9×10⁵ cells/m² are administered. In certain embodiments, about 1×10⁶ cells/m² are administered. In certain embodiments, about 2×10⁶ cells/m² are administered. In certain embodiments, about 3×10⁶ cells/m² are administered. In certain embodiments, about 4×10⁶ cells/m² are administered. In certain embodiments, about 5×10⁶ cells/m² are administered. In certain embodiments, about 6×10⁶ cells/m² are administered. In certain embodiments, about 7×10⁶ cells/m² are administered. In certain embodiments, about 8×10⁶ cells/m² are administered. In certain embodiments, about 9×10⁶ cells/m² are administered. In certain embodiments, about 1×10⁷ cells/m² are administered. In certain embodiments, about 2×10⁷ cells/m² are administered. In certain embodiments, about 3×10⁷ cells/m² are administered. In certain embodiments, about 4×10⁷ cells/m² are administered. In certain embodiments, about 5×10⁷ cells/m² are administered. In certain embodiments, about 6×10⁷ cells/m² are administered. In certain embodiments, about 7×10⁷ cells/m² are administered. In certain embodiments, about 8×10⁷ cells/m² are administered. In certain embodiments, about 9×10⁷ cells/m² are administered.

In certain embodiments, CAR T cells are administered once a day. In certain embodiments, CAR T cells are administered once a week. In certain embodiments, CAR T cells are administered once a month. In certain embodiments, CAR T cells are administered twice a week. In certain embodiments, CAR T cells are administered twice a month. In certain embodiments, CAR T cells are administered thrice a week. In certain embodiments, CAR T cells are administered thrice a month. In certain embodiments, CAR T cells are administered 4 times a month. In certain embodiments, the CAR T cells are administered as a single dose.

Another strategy deployed in cell-based therapeutics is to render a T-cell population transgenic for a recombinant T-cell receptor (TCR) specific for a TAA. Much like with CAR based therapies T cells that have been expanded in culture are transfected and transduced with a TAA specific TCR. In most cases this is with patient autologous cells that have been expanded in culture. In certain embodiments, the cell-based therapy is a T cell or T-cell population expressing a recombinant TCR. The TCR can be specific for a TAA, such as, glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, MART-1, Lewis Y antigen (LeY), tyrosinase and GP 100, prostatic acid phosphatase (PAP) prostate-specific antigen (PSA), ROR1, MUC16, CD171 (LICAM), B-cell maturation antigen (BCMA), WT1, HER-2/Neu/ErbB-2, CD19, CD20, or CD37.

In certain embodiments, the recombinant TCR T cells are administered by i.v. infusion. In certain embodiments, about 1×10⁵ cells/m² are administered. In certain embodiments, about 2×10⁵ cells/m² are administered. In certain embodiments, about 3×10⁵ cells/m² are administered. In certain embodiments, about 4×10⁵ cells/m² are administered. In certain embodiments, about 5×10⁵ cells/m² are administered. In certain embodiments, about 6×10⁵ cells/m² are administered. In certain embodiments, about 7×10⁵ cells/m² are administered. In certain embodiments, about 8×10⁵ cells/m² are administered. In certain embodiments, about 9×10⁵ cells/m² are administered. In certain embodiments, about 1×10⁶ cells/m² are administered. In certain embodiments, about 2×10⁶ cells/m² are administered. In certain embodiments, about 3×10⁶ cells/m² are administered. In certain embodiments, about 4×10⁶ cells/m² are administered. In certain embodiments, about 5×10⁶ cells/m² are administered. In certain embodiments, about 6×10⁶ cells/m² are administered. In certain embodiments, about 7×10⁶ cells/m² are administered. In certain embodiments, about 8×10⁶ cells/m² are administered. In certain embodiments, about 9×10⁶ cells/m² are administered. In certain embodiments, about 1×10⁷ cells/m² are administered. In certain embodiments, about 2×10⁷ cell s/m² are administered. In certain embodiments, about 3×10⁷ cells/m² are administered. In certain embodiments, about 4×10⁷ cells/m² are administered. In certain embodiments, about 5×10⁷ cells/m² are administered. In certain embodiments, about 6×10⁷ cells/m² are administered. In certain embodiments, about 7×10⁷ cells/m² are administered. In certain embodiments, about 8×10⁷ cell s/m² are administered. In certain embodiments, about 9×10⁷ cells/m² are administered.

In certain embodiments, the recombinant TCR T cells are administered once a day. In certain embodiments, the recombinant TCR T cells are administered once a week. In certain embodiments, the recombinant TCR T cells are administered once a month. In certain embodiments, the recombinant TCR T cells are administered twice a week. In certain embodiments, the recombinant TCR T cells are administered twice a month. In certain embodiments, the recombinant TCR T cells are administered thrice a week. In certain embodiments, the recombinant TCR T cells are administered thrice a month. In certain embodiments, the recombinant TCR T cells are administered 4 times a month.

In vitro treatments of T cells or T cell lines with HDACi can be combined with additional agents such as proliferative or pro-maintenance factors such as the cytokines IL-15, IL-7, or a combination thereof. In certain embodiments, in vitro treatments of T cells or T cell lines with nanatinostat can be combined with additional agents such as proliferative or pro-maintenance factors such as the cytokines IL-15, IL-7, or a combination thereof. In certain embodiments, the concentration of IL-15 comprises about 1 ng/mL to about 100 ng/mL. In certain embodiments, the concentration of IL-15 comprises about 1 ng/mL to about 5 ng/mL, about 1 ng/mL to about 10 ng/mL, about 1 ng/mL to about 20 ng/mL, about 1 ng/mL to about 30 ng/mL, about 1 ng/mL to about 40 ng/mL, about 1 ng/mL to about 50 ng/mL, about 1 ng/mL to about 60 ng/mL, about 1 ng/mL to about 70 ng/mL, about 1 ng/mL to about 80 ng/mL, about 1 ng/mL to about 90 ng/mL, about 1 ng/mL to about 100 ng/mL, about 5 ng/mL to about 10 ng/mL, about 5 ng/mL to about 20 ng/mL, about 5 ng/mL to about 30 ng/mL, about 5 ng/mL to about 40 ng/mL, about 5 ng/mL to about 50 ng/mL, about 5 ng/mL to about 60 ng/mL, about 5 ng/mL to about 70 ng/mL, about 5 ng/mL to about 80 ng/mL, about 5 ng/mL to about 90 ng/mL, about 5 ng/mL to about 100 ng/mL, about 10 ng/mL to about 20 ng/mL, about 10 ng/mL to about 30 ng/mL, about 10 ng/mL to about 40 ng/mL, about 10 ng/mL to about 50 ng/mL, about 10 ng/mL to about 60 ng/mL, about 10 ng/mL to about 70 ng/mL, about 10 ng/mL to about 80 ng/mL, about 10 ng/mL to about 90 ng/mL, about 10 ng/mL to about 100 ng/mL, about 20 ng/mL to about 30 ng/mL, about 20 ng/mL to about 40 ng/mL, about 20 ng/mL to about 50 ng/mL, about 20 ng/mL to about 60 ng/mL, about 20 ng/mL to about 70 ng/mL, about 20 ng/mL to about 80 ng/mL, about 20 ng/mL to about 90 ng/mL, about 20 ng/mL to about 100 ng/mL, about 30 ng/mL to about 40 ng/mL, about 30 ng/mL to about 50 ng/mL, about 30 ng/mL to about 60 ng/mL, about 30 ng/mL to about 70 ng/mL, about 30 ng/mL to about 80 ng/mL, about 30 ng/mL to about 90 ng/mL, about 30 ng/mL to about 100 ng/mL, about 40 ng/mL to about 50 ng/mL, about 40 ng/mL to about 60 ng/mL, about 40 ng/mL to about 70 ng/mL, about 40 ng/mL to about 80 ng/mL, about 40 ng/mL to about 90 ng/mL, about 40 ng/mL to about 100 ng/mL, about 50 ng/mL to about 60 ng/mL, about 50 ng/mL to about 70 ng/mL, about 50 ng/mL to about 80 ng/mL, about 50 ng/mL to about 90 ng/mL, about 50 ng/mL to about 100 ng/mL, about 60 ng/mL to about 70 ng/mL, about 60 ng/mL to about 80 ng/mL, about 60 ng/mL to about 90 ng/mL, about 60 ng/mL to about 100 ng/mL, about 70 ng/mL to about 80 ng/mL, about 70 ng/mL to about 90 ng/mL, about 70 ng/mL to about 100 ng/mL, about 80 ng/mL to about 90 ng/mL, about 80 ng/mL to about 100 ng/mL, or about 90 ng/mL to about 100 ng/mL. In certain embodiments, the concentration of IL-15 comprises about 1 ng/mL, about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, or about 100 ng/mL. In certain embodiments, the concentration of IL-15 comprises at least about 1 ng/mL, about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, or about 90 ng/mL. In certain embodiments, the concentration of IL-15 comprises at most about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, or about 100 ng/mL. In certain embodiments, IL-15 is combined with IL-7 at a concentration of about 1 ng/mL, 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, or about 100 ng/mL.

In vitro treatments of T cells or T cell lines with HDACi can be combined with additional immunotherapeutic agents that are checkpoint inhibitors, such as antagonistic antibodies against PD-1, PD-L1, or PD-L2 and combinations thereof. In certain embodiments, the checkpoint inhibitor antibody comprises Ipilimumab, Pembrolizumab, Nivolumab, Spartalizumab, Atezolizumab, Avelumab, or Durvalumab. The checkpoint inhibitor antibody can optionally be included with an amount of IL-15 or IL-7 either in the same or a different contact step. In certain embodiments, the concentration of checkpoint inhibitor antibody contacted with a T cell-based immunotherapy in the method is about 10 micrograms/mL to about 100 micrograms/mL. In certain embodiments, the concentration of checkpoint inhibitor antibody contacted with a T cell based immunotherapy in the method is about 10 micrograms/mL to about 20 micrograms/mL, about 10 micrograms/mL to about 30 micrograms/mL, about 10 micrograms/mL to about 40 micrograms/mL, about 10 microgram s/mL to about 50 microgram s/mL, about 10 microgram s/mL to about 60 microgram s/mL, about 10 microgram s/mL to about 70 microgram s/mL, about 10 microgram s/mL to about 80 microgram s/mL, about 10 microgram s/mL to about 90 microgram s/mL, about 10 microgram s/mL to about 100 microgram s/mL, about 20 microgram s/mL to about 30 microgram s/mL, about 20 microgram s/mL to about 40 microgram s/mL, about 20 microgram s/mL to about 50 microgram s/mL, about 20 micrograms/mL to about 60 micrograms/mL, about 20 micrograms/mL to about 70 microgram s/mL, about 20 microgram s/mL to about 80 microgram s/mL, about 20 micrograms/mL to about 90 micrograms/mL, about 20 micrograms/mL to about 100 microgram s/mL, about 30 microgram s/mL to about 40 microgram s/mL, about 30 microgram s/mL to about 50 microgram s/mL, about 30 microgram s/mL to about 60 microgram s/mL, about 30 microgram s/mL to about 70 microgram s/mL, about 30 microgram s/mL to about 80 microgram s/mL, about 30 microgram s/mL to about 90 microgram s/mL, about 30 microgram s/mL to about 100 microgram s/mL, about 40 microgram s/mL to about 50 microgram s/mL, about 40 microgram s/mL to about 60 microgram s/mL, about 40 microgram s/mL to about 70 microgram s/mL, about 40 microgram s/mL to about 80 microgram s/mL, about 40 microgram s/mL to about 90 microgram s/mL, about 40 microgram s/mL to about 100 microgram s/mL, about 50 microgram s/mL to about 60 microgram s/mL, about 50 microgram s/mL to about 70 microgram s/mL, about 50 microgram s/mL to about 80 microgram s/mL, about 50 microgram s/mL to about 90 microgram s/mL, about 50 microgram s/mL to about 100 microgram s/mL, about 60 microgram s/mL to about 70 micrograms/mL, about 60 microgram s/mL to about 80 microgram s/mL, about 60 microgram s/mL to about 90 microgram s/mL, about 60 microgram s/mL to about 100 microgram s/mL, about 70 micrograms/mL to about 80 micrograms/mL, about 70 micrograms/mL to about 90 microgram s/mL, about 70 microgram s/mL to about 100 microgram s/mL, about 80 microgram s/mL to about 90 microgram s/mL, about 80 microgram s/mL to about 100 micrograms/mL, or about 90 micrograms/mL to about 100 micrograms/mL. In certain embodiments, the concentration of checkpoint inhibitor antibody contacted with a T cell-based immunotherapy in the method is about 10 micrograms/mL, about 20 micrograms/mL, about 30 microgram s/mL, about 40 microgram s/mL, about 50 micrograms/mL, about 60 microgram s/mL, about 70 micrograms/mL, about 80 micrograms/mL, about 90 micrograms/mL, or about 100 micrograms/mL. In certain embodiments, the concentration of checkpoint inhibitor antibody contacted with a T cell-based immunotherapy in the method is at least about 10 micrograms/mL, about 20 microgram s/mL, about 30 microgram s/mL, about 40 microgram s/mL, about 50 micrograms/mL, about 60 micrograms/mL, about 70 micrograms/mL, about 80 micrograms/mL, or about 90 micrograms/mL. In certain embodiments, the concentration of checkpoint inhibitor antibody contacted with a T cell-based immunotherapy in the method is at most about 20 micrograms/mL, about 30 micrograms/mL, about 40 micrograms/mL, about 50 micrograms/mL, about 60 micrograms/mL, about 70 micrograms/mL, about 80 micrograms/mL, about 90 micrograms/mL, or about 100 micrograms/mL.

### NK Cell Based Therapies

Natural killer (NK) cells can also be employed in cell-based therapies. NK cells are innate lymphocytic immune cells that display cytotoxic activity. As with T cells an NK cell can be transduced with a CAR (creating a CAR NK cell) or used as a primary population without transduction. CAR NK cells can be established from a primary autologous population or using an NK cell line. Common NK cell lines that can be used are the NK-92 cell line (available from the ATCC; CRL-2497), or the KHYG-1 cell line. In certain embodiments, the engineered NK cell line is made from the KHYG-1 cell line. See Yagita et al., “A novel natural killer cell line (KHYG-1) from a patient with aggressive natural killer cell leukemia carrying a p53 point mutation.” Leukemia 14(5):922-30. The NK cells for use with the HDACi of the current disclosure can be made from any NK cell population including primary cells or established cell lines. In certain embodiments, the NK cell is a human NK cell. Primary natural killer cells in humans express the cell surface marker CD56, and in certain embodiments, the engineered natural killer cells can be produced from CD56 positive cells as determined, by way of non-limiting example, by flow cytometry. In certain embodiments, the natural killer cell can be from an autologous, or from a heterologous source. The NK cell can be isolated from the peripheral blood of a donor or the individual to be treated using a method such as cell sorting or magnetic beads. NK cells isolated from a donor can be expanded ex vivo by culturing in interleukin-2 and interleukin-15 for greater than 7 days. NK-cell populations can also be derived from hematopoietic stem cells (HSCs) or induced pluripotent stem cells (iPSCs) using methods known in the art. In certain embodiments, T-cell populations are derived/differentiated from iPSCs. The source of the iPSCs can be either autologous or heterologous. In certain embodiments, T-cell populations are derived/differentiated from (HSCs) cells. The source of the HSCs can be either autologous or heterologous. NK-cell populations can be marked by CD56 expression. In certain embodiments, an NK-cell population useful with the media and methods described herein will be at least 60%, 70%, 80%, 90%, or 95% positive for CD56 by FACS staining.

The NK cell or NK-cell population expressing a CAR can express a car specific for a TAA such as, glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, MART-1, Lewis Y antigen (LeY), tyrosinase and GP 100, prostatic acid phosphatase (PAP) prostate-specific antigen (PSA), ROR1, MUC16, CD171 (LICAM), B-cell maturation antigen (BCMA), WT1, HER-2/Neu/ErbB-2, CD19, CD20, or CD37.

In certain embodiments, the CAR NK cells are administered by i.v. infusion. In certain embodiments, about 1×10⁵ cells/m² are administered. In certain embodiments, about 2×10⁵ cells/m² are administered. In certain embodiments, about 3×10⁵ cells/m² are administered. In certain embodiments, about 4×10⁵ cells/m² are administered. In certain embodiments, about 5×10⁵ cells/m² are administered. In certain embodiments, about 6×10⁵ cells/m² are administered. In certain embodiments, about 7×10⁵ cells/m² are administered. In certain embodiments, about 8×10⁵ cells/m² are administered. In certain embodiments, about 9×10⁵ cells/m² are administered. In certain embodiments, about 1×10⁶ cells/m² are administered. In certain embodiments, about 2×10⁶ cells/m² are administered. In certain embodiments, about 3×10⁶ cells/m² are administered. In certain embodiments, about 4×10⁶ cells/m² are administered. In certain embodiments, about 5×10⁶ cells/m² are administered. In certain embodiments, about 6×10⁶ cells/m² are administered. In certain embodiments, about 7×10⁶ cells/m² are administered. In certain embodiments, about 8×10⁶ cells/m² are administered. In certain embodiments, about 9×10⁶ cells/m² are administered. In certain embodiments, about 1×10⁷ cells/m² are administered. In certain embodiments, about 2×10⁷ cells/m² are administered. In certain embodiments, about 3×10⁷ cells/m² are administered. In certain embodiments, about 4×10⁷ cells/m² are administered. In certain embodiments, about 5×10⁷ cells/m² are administered. In certain embodiments, about 6×10⁷ cells/m² are administered. In certain embodiments, about 7×10⁷ cells/m² are administered. In certain embodiments, about 8×10⁷ cells/m² are administered. In certain embodiments, about 9×10⁷ cells/m² are administered.

In certain embodiments, CAR NK cells are administered once a day. In certain embodiments, CAR NK cells are administered once a week. In certain embodiments, CAR NK cells are administered once a month. In certain embodiments, CAR NK cells are administered twice a week. In certain embodiments, CAR NK cells are administered twice a month. In certain embodiments, CAR NK cells are administered thrice a week. In certain embodiments, CAR NK cells are administered thrice a month. In certain embodiments, CAR NK cells are administered 4 times a month. In certain embodiments, the CAR NK cells are administered as a single dose.

Additionally, NK cells can be engineered to express high-affinity Fc receptors (HaNK) and combined with a tumor targeting antibody to target killing of Tumor cells in vivo. For example, CD16 is a high affinity Fc receptor that will bind an antibody through its Fc portion allowing the Fab portion free to interact with a tumor cell, thus recruiting the cytotoxic NK cell to a tumor site. NK cells modified with high-affinity Fc receptors are described, for example, in U.S. Pat. Nos. 7,618,817 and 8,313,943 which are incorporated herein in their entirety. An NK cell expressing a high affinity Fc receptor can be combined with a TAA specific antibody such as the monoclonal antibody is Lambrolizumab, Dupilumab, Tabalumab, Galiximab, Pritumumab, Trastuzumab, Amatuximab, Coltuximab ravtansine, Ensituximab, Indatuximab ravtansine, Isatuximab, Mirvetuximab soravtansine, Siltuxima, Ublituximab, Zatuximab, Ontuxizumab, Pasotuxizumab, Anetumab ravtansine, Ascrinvacumab, Conatumumab, Daratumumab, Durvalumab, Dusigitumab, Elgemtumab, Ganitumab, Imalumab, Indusatumab vedotin, Lexatumumab, Mapatumumab, Narnatumab, Nesvacumab, Nivolumab, Olaratum, Parsatuzumab, Patritumab, Radretumab, Robatumuma, Seribantumab, Tarextumab, Ticilimumab (tremelimumab), Tovetumab, Tremelimumab, Vantictumab, Abituzumab, Alacizumab pegol, Atezolizumab, cBR96-doxorubicin immunoconjugate, Codrituzumab, Demcizumab, Denintuzumab mafodotin, Emactuzumab, Emibetuzumab, Enoblituzumab, Imgatuzumab, Inotuzumab ozogamicin, Lifastuzumab vedotin, Lintuzuma, Lorvotuzumab mertansin, Lumretuzumab, Margetuximab, Mogamulizumab, Ocaratuzumab, Onartuzumab, Oportuzumab monatox, Otlertuzumab, Pertuzumab, Pinatuzumab vedotin, Polatuzumab vedotin, Sacituzumab govitecan, Samalizumab, Sibrotuzumab, Tacatuzumab tetraxetan, Tigatuzumab, Tucotuzumab celmoleukin, Vandortuzumab vedotin, Vanucizumab, Vorsetuzumab mafodotin, Pidilizumab, Drozitumab, Icrucumab, Urelumab, Dalotuzumab, Enavatuzumab, Ficlatuzumab, Pembrolizumab, Enfortumab vedotin, Bavituximab, Epratuzumab, Cantuzumab ravtansine, Sonepcizumab, Tuvirumab, Lumiliximab, Ofatumumab, TGN1412, Girentuximab, Panitumumab, Labetuzumab, Cantuzumab mertansine, Votumumab, Matuzumab, Regavirumab, Sevirumab, Otelixizumab, IMAB362, Brentuximab vedotin, Dacetuzumab, Ulocuplumab, Teprotumumab, Apolizumab, Atorolimumab, Iratumumab, TNX-650, Afutuzumab, Rituximab, Ecromeximab, TRBS07, Flanvotumab, Ipilimumab, Glembatumumab vedotin, Etaracizumab, Bevacizumab, Cetuximab, Elotuzumab, Milatuzumab, Lucatumumab, Dinutuximab, Belimumab, Veltuzumab, Necitumumab, Carlumab, Romosozumab, Denosumab, Farletuzumab, Pankomab, Sofituzumab vedotin, Citatuzumab bogatox, Clivatuzumab tetraxetan, Abciximab, Daclizumab, Basiliximab, Adecatumumab, Derlotuximab biotin, Ruplizumab, Clenoliximab, Canakinumab, Fletikumab, Mavrilimumab, Sirukumab, ALD518, Atlizumab (tocilizumab), Clazakizumab, Infliximab, Ocrelizumab, Zanolimumab, Golimumab, Sarilumab, Adalimumab, Fezakinumab, Volociximab, Cixutumumab, Ramucirumab, Rilotumumab, Intetumumab, Bivatuzumab mertansine, Zalutumumab, Nimotuzumab, Anifrolumab, Rontalizumab, Metelimumab, Alemtuzumab, or Pateclizumab. In certain embodiments, the monoclonal antibody is BS-936559, MSB0010718C, or MEDI4736.

In certain embodiments, the HaNK cells are administered by i.v. infusion. The HaNK cells can be complexed with an antibody before administration (before, during, or after HDACi treatment), or administered after a TAA specific antibody. In certain embodiments, about 1×10⁵ cells/m² are administered. In certain embodiments, about 2×10⁵ cells/m² are administered. In certain embodiments, about 3×10⁵ cells/m² are administered. In certain embodiments, about 4×10⁵ cells/m² are administered. In certain embodiments, about 5×10⁵ cells/m² are administered. In certain embodiments, about 6×10⁵ cells/m² are administered. In certain embodiments, about 7×10⁵ cells/m² are administered. In certain embodiments, about 8×10⁵ cells/m² are administered. In certain embodiments, about 9×10⁵ cells/m² are administered. In certain embodiments, about 1×10⁶ cells/m² are administered. In certain embodiments, about 2×10⁶ cells/m² are administered. In certain embodiments, about 3×10⁶ cells/m² are administered. In certain embodiments, about 4×10⁶ cells/m² are administered. In certain embodiments, about 5×10⁶ cells/m² are administered. In certain embodiments, about 6×10⁶ cells/m² are administered. In certain embodiments, about 7×10⁶ cells/m² are administered. In certain embodiments, about 8×10⁶ cells/m² are administered. In certain embodiments, about 9×10⁶ cells/m² are administered. In certain embodiments, about 1×10⁷ cells/m² are administered. In certain embodiments, about 2×10⁷ cells/m² are administered. In certain embodiments, about 3×10⁷ cells/m² are administered. In certain embodiments, about 4×10⁷ cells/m² are administered. In certain embodiments, about 5×10⁷ cells/m² are administered. In certain embodiments, about 6×10⁷ cells/m² are administered. In certain embodiments, about 7×10⁷ cells/m² are administered. In certain embodiments, about 8×10⁷ cells/m² are administered. In certain embodiments, about 9×10⁷ cells/m² are administered.

In certain embodiments, HaNK cells are administered once a day. In certain embodiments, HaNK cells are administered once a week. In certain embodiments, HaNK cells are administered once a month. In certain embodiments, HaNK cells are administered twice a week. In certain embodiments, HaNK cells are administered twice a month. In certain embodiments, HaNK cells are administered thrice a week. In certain embodiments, HaNK cells are administered thrice a month. In certain embodiments, HaNK cells are administered 4 times a month. In certain embodiments, the HaNK cells are administered as a single dose.

NK-cell populations and NK-cell lines used in the method described herein, (including CAR NK and HaNK cells) display augmented functionality. This functionality can be a physiological function such as increased half-life in the circulation, higher trafficking to tumor sites, increased cytotoxic activity, or increased cytokine/chemokine secretion compared to a non-HDACi treated NK-cell population. In certain embodiments, the HDACi treated cell population or cell line exhibits a half-life that is 10%, 25%, 50%, or 75% longer than a non-HDACi treated cell population or cell line. In certain embodiments, the HDACi treated cell population or cell line exhibits a half-life that is 2-fold, 3-fold, 4-fold, or 5-fold longer than a non-HDACi treated cell population or cell line. In certain embodiments, the HDACi treated cell population or cell line exhibits trafficking to a tumor site that is 10%, 25%, 50%, or 75% greater than a non-HDACi treated cell population or cell line. In certain embodiments, the HDACi treated cell population or cell line exhibits trafficking to a tumor site that is 2-fold, 3-fold, 4-fold, or 5-fold greater than a non-HDACi treated cell population or cell line. In certain embodiments, the HDACi treated cell population or cell line exhibits cytotoxic activity that is 10%, 25%, 50%, or 75% greater than a non-HDACi treated cell population or cell line. In certain embodiments, the HDACi treated cell population or cell line exhibits cytotoxic activity that is 2-fold, 3-fold, 4-fold, or 5-fold greater than a non-HDACi treated cell population or cell line. In certain embodiments, the HDACi treated cell population or cell line releases IFN-γ at a level 10%, 25%, 50%, or 75% greater than a non-HDACi treated cell population or cell line. In certain embodiments, the HDACi treated cell population or cell line releases IFN-γ at a level 2-fold, 3-fold, 4-fold, or 5-fold greater than a non-HDACi treated cell population or cell line. In certain embodiments, the HDACi treated cell population or cell line releases TRAIL at a level 10%, 25%, 50%, or 75% greater than a non-HDACi treated cell population or cell line. In certain embodiments, the HDACi treated cell population or cell line releases TRAIL at a level 2-fold, 3-fold, 4-fold, or 5-fold greater than a non-HDACi treated cell population or cell line. Alternatively, the increased functionality seen in an NK-cell population or NK-cell line can be a cellular or molecular function, such as increased expression of an activated cell-marker, reduced expression of an inhibitory cell-marker, increased cell-surface expression of an activated cell-marker, or reduced expression of an inhibitory cell-marker compared to a non-HDACi treated NK-cell population. FasL is a cell-surface receptor that is expressed on NK cells and contributes to cytotoxicity. In certain embodiments, the HDACi treated cell population or cell line expresses FasL at the cell-surface at a level that is 10%, 25%, 50%, or 75% greater than a non-HDACi treated cell population or cell line. In certain embodiments, the HDACi treated cell population or cell line expresses FasL at the cell-surface at a level that is 2-fold, 3-fold, 4-fold, or 5-fold greater than a non-HDACi treated cell population or cell line. KLRC2 is a transcription factor that is associated with NK-cell cytotoxicity. In certain embodiments, the HDACi treated cell population or cell line expresses KLRC2 at a level that is 10%, 25%, 50%, or 75% greater than a non-HDACi treated cell population or cell line. In certain embodiments, the HDACi treated cell population or cell line expresses KLRC2 at a level that is 2-fold, 3-fold, 4-fold, or 5-fold greater than a non-HDACi treated cell population or cell line or cell line. In certain embodiments, the HDACi treated cell population or cell line expresses IFN-γ mRNA at a level 10%, 25%, 50%, or 75% greater than a non-HDACi treated cell population or cell line. In certain embodiments, the HDACi treated cell population or cell line expresses IFN-γ mRNA at a level 2-fold, 3-fold, 4-fold, 5-fold, or 10-fold greater than a non-HDACi treated cell population or cell line. In certain embodiments, the HDACi treated cell population or cell line perforin mRNA at a level 10%, 25%, 50%, or 75% greater than a non-HDACi treated cell population or cell line. In certain embodiments, the HDACi treated cell population or cell line expresses perforin mRNA at a level 2-fold, 3-fold, 4-fold, 5-fold, or 10-fold greater than a non-HDACi treated cell population or cell line. In certain embodiments, the HDACi treated cell population or cell line expresses granzymeB mRNA at a level 10%, 25%, 50%, or 75% greater than a non-HDACi treated cell population or cell line. In certain embodiments, the HDACi treated cell population or cell line expresses granzymeB mRNA at a level 2-fold, 3-fold, 4-fold, 5-fold, or 10-fold greater than a non-HDACi treated cell population or cell line. A non-HDACi treated cell population or cell line can for example, be a comparison of a before and after treatment or comparison to a similarly treated cell population except for HDACi treatment.

In vitro treatments of NK cells with HDACi can be combined with additional agents such as proliferative or pro-maintenance factors such as the cytokines IL-15, IL-7, or a combination thereof. In certain embodiments, in vitro treatments of NK cells with nanatinostat can be combined with additional agents such as proliferative or pro-maintenance factors such as the cytokines IL-15, IL-7, or a combination thereof. In certain embodiments, the concentration of IL-15 comprises about 1 ng/mL to about 100 ng/mL. In certain embodiments, the concentration of IL-15 comprises about 1 ng/mL to about 5 ng/mL, about 1 ng/mL to about 10 ng/mL, about 1 ng/mL to about 20 ng/mL, about 1 ng/mL to about 30 ng/mL, about 1 ng/mL to about 40 ng/mL, about 1 ng/mL to about 50 ng/mL, about 1 ng/mL to about 60 ng/mL, about 1 ng/mL to about 70 ng/mL, about 1 ng/mL to about 80 ng/mL, about 1 ng/mL to about 90 ng/mL, about 1 ng/mL to about 100 ng/mL, about 5 ng/mL to about 10 ng/mL, about 5 ng/mL to about 20 ng/mL, about 5 ng/mL to about 30 ng/mL, about 5 ng/mL to about 40 ng/mL, about 5 ng/mL to about 50 ng/mL, about 5 ng/mL to about 60 ng/mL, about 5 ng/mL to about 70 ng/mL, about 5 ng/mL to about 80 ng/mL, about 5 ng/mL to about 90 ng/mL, about 5 ng/mL to about 100 ng/mL, about 10 ng/mL to about 20 ng/mL, about 10 ng/mL to about 30 ng/mL, about 10 ng/mL to about 40 ng/mL, about 10 ng/mL to about 50 ng/mL, about 10 ng/mL to about 60 ng/mL, about 10 ng/mL to about 70 ng/mL, about 10 ng/mL to about 80 ng/mL, about 10 ng/mL to about 90 ng/mL, about 10 ng/mL to about 100 ng/mL, about 20 ng/mL to about 30 ng/mL, about 20 ng/mL to about 40 ng/mL, about 20 ng/mL to about 50 ng/mL, about 20 ng/mL to about 60 ng/mL, about 20 ng/mL to about 70 ng/mL, about 20 ng/mL to about 80 ng/mL, about 20 ng/mL to about 90 ng/mL, about 20 ng/mL to about 100 ng/mL, about 30 ng/mL to about 40 ng/mL, about 30 ng/mL to about 50 ng/mL, about 30 ng/mL to about 60 ng/mL, about 30 ng/mL to about 70 ng/mL, about 30 ng/mL to about 80 ng/mL, about 30 ng/mL to about 90 ng/mL, about 30 ng/mL to about 100 ng/mL, about 40 ng/mL to about 50 ng/mL, about 40 ng/mL to about 60 ng/mL, about 40 ng/mL to about 70 ng/mL, about 40 ng/mL to about 80 ng/mL, about 40 ng/mL to about 90 ng/mL, about 40 ng/mL to about 100 ng/mL, about 50 ng/mL to about 60 ng/mL, about 50 ng/mL to about 70 ng/mL, about 50 ng/mL to about 80 ng/mL, about 50 ng/mL to about 90 ng/mL, about 50 ng/mL to about 100 ng/mL, about 60 ng/mL to about 70 ng/mL, about 60 ng/mL to about 80 ng/mL, about 60 ng/mL to about 90 ng/mL, about 60 ng/mL to about 100 ng/mL, about 70 ng/mL to about 80 ng/mL, about 70 ng/mL to about 90 ng/mL, about 70 ng/mL to about 100 ng/mL, about 80 ng/mL to about 90 ng/mL, about 80 ng/mL to about 100 ng/mL, or about 90 ng/mL to about 100 ng/mL. In certain embodiments, the concentration of IL-15 comprises about 1 ng/mL, about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, or about 100 ng/mL. In certain embodiments, the concentration of IL-15 comprises at least about 1 ng/mL, about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, or about 90 ng/mL. In certain embodiments, the concentration of IL-15 comprises at most about 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, or about 100 ng/mL. In certain embodiments, IL-15 is combined with IL-7 at a concentration of about 1 ng/mL, 5 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, or about 100 ng/mL.

### Medium

Also disclosed herein is cell culture medium useful for augmenting a cell-based immunotherapy. In certain embodiments, the culture medium lacks serum of human or animal origin. In certain embodiment the medium comprises a class I HDACi. In some embodiments, the class I HDACi is Nanatinostat. In certain embodiments, the HDAC is present at a concentration that increases histone acetylation in a cell-based immunotherapeutic. In a certain embodiment, the histone with increased acetylation comprises Histone H3. In a certain embodiment, the histone with increased acetylation comprises Histone H3 and the increased acetylation is at lysine 9. In certain embodiments, the cell culture medium comprises nanatinostat at a concentration of less than about 10 μM. In certain embodiments, the cell culture medium comprises nanatinostat at a concentration of less than about 5 μM. In certain embodiments, the cell culture medium comprises nanatinostat at a concentration of less than about 2 μM. In certain embodiments, the cell culture medium comprises nanatinostat at a concentration of less than about 1 μM. In certain embodiments, the cell culture medium comprises nanatinostat at a concentration of less than about 900 nM. In certain embodiments, the cell culture medium comprises nanatinostat at a concentration of less than about 800 nM. In certain embodiments, the cell culture medium comprises nanatinostat at a concentration of less than about 700 nM. In certain embodiments, the cell culture medium comprises nanatinostat at a concentration of less than about 600 nM. In certain embodiments, the cell culture medium comprises nanatinostat at a concentration of less than about 500 nM. In certain embodiments, the cell culture medium comprises nanatinostat at a concentration of less than about 400 nM. In certain embodiments, the cell culture medium comprises nanatinostat at a concentration of less than about 300 nM. In certain embodiments, the cell culture medium comprises nanatinostat at a concentration of less than about 200 nM. In certain embodiments, the cell culture medium comprises nanatinostat at a concentration of less than about 100 nM. In certain embodiments, the cell culture medium comprises nanatinostat at a concentration of less than about 50 nM. In certain embodiments, the cell culture medium comprises nanatinostat at a concentration of greater than about 1 nM. In certain embodiments, the cell culture medium comprises nanatinostat at a concentration of greater than about 2 nM. In certain embodiments, the cell culture medium comprises nanatinostat at a concentration of greater than about 5 nM. In certain embodiments, the cell culture medium comprises nanatinostat at a concentration of greater than about 10 nM. In certain embodiments, the cell culture medium comprises nanatinostat at a concentration of greater than about 100 nM. In certain embodiments, the nanatinostat is present in the cell culture medium between about 1 nM and about 5 μM, between about 1 nM and about 2 μM, between about 1 nM and about 1 μM, between about 1 nM and about 900 nM, between about 1 nM and about 800 nM, between about 1 nM and about 700 nM, between about 1 nM and about 600 nM, between about 1 nM and about 500 nM, between about 1 nM and about 400 nM, between about 1 nM and about 300 nM, between about 1 nM and about 200 nM, between about 1 nM and about 100 nM, between about 1 nM and about 50 nM, between about 1 nM and about 25 nM, between about 10 nM and about 5 μM, between about 10 nM and about 2 μM, between about 10 nM and about 1 μM, between about 10 nM and about 900 nM, between about 10 nM and about 800 nM, between about 10 nM and about 700 nM, between about 10 nM and about 600 nM, between about 10 nM and about 500 nM, between about 10 nM and about 400 nM, between about 10 nM and about 300 nM, between about 10 nM and about 200 nM, between about 1 nM and about 100 nM, between about 10 nM and about 50 nM, between about 10 nM and about 25 nM. The medium herein can consist essentially of the HDACi included and the medium, without additional cytokines, chemokines, or growth factors that contribute to augmentation of a cell-based therapy.

The cell culture can be provided lyophilized for reconstitution with sterile distilled water, in a suitable container as a concentrated solution (e.g., 10× or 100×), or undiluted. The medium can be supplied as a kit with suitable reagents for T cell or NK cell isolation or expansion. The medium can be supplied as a kit with HDACi and medium in separate containers. The medium can be supplied as a kit with nanatinostat and medium in separate containers.

Specific embodiments of a cell culture medium are now described.
1. A cell culture media comprising an HDAC inhibitor (HDACi), wherein the HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide).
2. The cell culture media of embodiment 1, which does not comprise serum of non-human origin.
3. The cell culture media of embodiment 1, which does not comprise serum.
4. The cell culture media of any one of embodiment 1 to 3, further comprising a cell-based immunotherapy.
5. The cell culture media of embodiment 4, wherein the cell-based immunotherapy comprises a T-cell population.
6. The cell culture media of embodiment 5, wherein the T-cell comprises a primary T-cell population derived from a healthy individual.
7. The cell culture media of embodiment 5, wherein the T-cell comprises a primary T-cell population derived from an individual afflicted with a disease.
8. The cell culture media of embodiment 5, wherein the T-cell comprises a primary T-cell population derived from the individual afflicted with the disease.
9. The cell culture media of any one of embodiments 5 to 8, wherein the T-cell population further comprises a chimeric antigen receptor (CAR).
10. The cell culture media of any one of embodiments 5 to 9, wherein the cell culture media further comprises a tumor associated antigen.
11. The cell culture media of any one of embodiments 5 to 9, wherein the cell culture media further comprises a pro-inflammatory cytokine.
12. The cell culture media of any one of embodiments 5 to 9, wherein the T-cell population is enriched for CD4 positive T cells.
13. The cell culture media of any one of embodiments 5 to 9, wherein the T-cell population is enriched for CD8 positive T cells.
14. The cell culture media of any one of embodiments 5 to 13, wherein FoxP3 expression is reduced in the T-cell population after contacting the cell-based immunotherapy with the cell culture media.
15. The cell culture media of any one of embodiments 5 to 13, wherein secretion of interferon gamma is increased in the T-cell population after contacting the cell-based immunotherapy with the cell culture media.
16. The cell culture media of any one of embodiments 5 to 13, wherein cell-surface expression of CXCR3 is increased in the T cell-population after contacting the cell-based immunotherapy with the cell culture media.
17. The cell culture media of embodiment 4, wherein the cell-based immunotherapy comprises a T-cell line.
18. The cell culture media of embodiment 17, wherein the T cell line comprises a chimeric antigen receptor.
19. The cell culture media of embodiments 17 or 18, wherein FoxP3 expression is reduced in the T cell line after contacting the cell-based immunotherapy with the cell culture media.
20. The cell culture media of embodiments 17 or 18, wherein secretion of interferon gamma is increased in the T cell line after contacting the cell-based immunotherapy with the cell culture media.
21. The cell culture media of embodiments 17 or 18, wherein cell-surface expression of CXCR3 is increased in the T cell line after contacting the cell-based immunotherapy with the cell culture media.
22. The cell culture media of embodiment 4, wherein the cell-based therapy comprises a natural killer cell line or primary natural killer cell population.
23. The cell culture media of embodiment 22, wherein the natural killer cell line or population comprises a chimeric antigen receptor.
24. The cell culture media of embodiment 22, wherein the natural killer cell line or population comprises a high-affinity Fc receptor.
25. The cell culture media of any one of embodiments 22 to 24, wherein secretion of interferon gamma is increased in the natural killer cell line or population after contacting the cell-based immunotherapy with the cell culture media.
26. The cell culture media of any one of embodiments 22 to 24, for use in a method of inhibiting or reversing T cell exhaustion.
27. The cell culture media of any one of embodiments 22 to 24, for use in a method of treating an individual afflicted with a disease.
28. The cell culture media of embodiment 27, wherein the disease is a cancer.
29. The cell culture media of embodiment 28, wherein the cancer is breast cancer, cervical cancer, colon cancer, head and neck cancer, kidney cancer, liver cancer, lung cancer, melanoma, ovarian cancer, pancreatic cancer, or prostate cancer.
30. The cell culture media of embodiment 29, wherein the cancer is a leukemia or lymphoma.

### Additional Agents

The HDACi can be combined in culture with an additional agent to augment a cell-based immunotherapy. In certain embodiments, the immunotherapeutic agent is a cytokine or chemokine. In certain embodiments, the cytokine is an interferon. In certain embodiments, the cytokine is interferon alpha. In certain embodiments, the cytokine is interferon beta. In certain embodiments, the cytokine is interferon gamma. In certain embodiments, the cytokine is an interleukin. In certain embodiments, the cytokine is interleukin 1. In certain embodiments, the cytokine is interleukin 2. In certain embodiments, the cytokine is interleukin 7. In certain embodiments, the cytokine is interleukin 15. In certain embodiments, the cytokine is a hematopoietic growth factor.

### Cancers

In certain embodiments, the methods of this disclosure are for the treatment of cancer or the manufacture of a medicament to treat cancer or a tumor. In certain embodiments, the methods of this disclosure are for augmenting the treatment of cancer or a tumor. In certain embodiments, the cancer or tumor is Acute Lymphoblastic Leukemia, Adult; Acute Lymphoblastic Leukemia, Childhood; Acute Myeloid Leukemia, Adult; Acute Myeloid Leukemia, Childhood; Adreno cortical Carcinoma; Adrenocortical Carcinoma, Childhood; Adolescents, Cancer in; AIDS-Related Cancers; AIDS-Related Lymphoma; Anal Cancer; Appendix Cancer; Astrocytomas, Childhood; Atypical Teratoid/Rhabdoid Tumor, Childhood, Central Nervous System; Basal Cell Carcinoma; Bile Duct Cancer, Extrahepatic; Bladder Cancer; Bladder Cancer, Childhood; Bone Cancer, Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Brain Tumor, Central Nervous System Embryonal Tumors, Childhood; Brain Tumor, Astro cytomas, Childhood; Brain Tumor, Craniopharyngioma, Childhood; Brain Tumor, Ependymoblastoma, Childhood; Brain Tumor, Ependymoma, Childhood; Brain Tumor, Medulloblastoma, Childhood; Brain Tumor, Medulloepithelioma, Childhood; Brain Tumor, Pineal Parenchymal Tumors of Intermediate Differentiation, Childhood; Brain Tumor, Supratentorial Primitive Neuro ectodermal Tumors and Pineoblastoma, Childhood; Brain and Spinal Cord Tumors, Childhood (Other); Breast Cancer; Breast Cancer and Pregnancy; Breast Cancer, Childhood; Breast Cancer, Male; Bronchial Tumors, Childhood; Burkitt Lymphoma; Carcinoid Tumor, Childhood; Carcinoid Tumor, Gastrointestinal; Carcinoma of Unknown Primary; Central Nervous System Atypical Teratoid/Rhabdoid Tumor, Childhood; Central Nervous System Embryonal Tumors, Childhood; Central Nervous System (CNS) Lymphoma, Primary; Cervical Cancer; Cervical Cancer, Childhood; Childhood Cancers; Chordoma, Childhood; Chronic Lymphocytic Leukemia; Chronic Myelogenous Leukemia; Chronic Myeloproliferative Disorders; Colon Cancer; Colorectal Cancer, Childhood; Craniopharyngioma, Childhood; Cutaneous T-Cell Lymphoma; Embryonal Tumors, Central Nervous System, Childhood; Endometrial Cancer; Ependymoblastoma, Childhood; Ependymoma, Childhood; Esophageal Cancer; Esophageal Cancer, Childhood; Esthesioneuroblastoma, Childhood; Ewing Sarcoma Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Gastrointestinal Stromal Tumor (GIST); Gastrointestinal Stromal Cell Tumor, Childhood; Germ Cell Tumor, Extracranial, Childhood; Germ Cell Tumor, Extragonadal; Germ Cell Tumor, Ovarian; Gestational Trophoblastic Tumor; Glioma, Adult; Glioma, Childhood Brain Stem; Hairy Cell Leukemia; Head and Neck Cancer; Heart Cancer, Childhood; Hepatocellular (Liver) Cancer, Adult (Primary); Hepatocellular (Liver) Cancer, Childhood (Primary); Histiocytosis, Langerhans Cell; Hodgkin Lymphoma, Adult; Hodgkin Lymphoma, Childhood; Hypopharyngeal Cancer; Intraocular Melanoma; Islet Cell Tumors (Endocrine Pancreas); Kaposi Sarcoma; Kidney (Renal Cell) Cancer; Kidney Cancer, Childhood; Langerhans Cell Histiocytosis; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin, Adult; Lymphoma, Hodgkin, Childhood; Lymphoma, Non-Hodgkin, Adult; Lymphoma, Non-Hodgkin, Childhood; Lymphoma, Primary Central Nervous System (CNS); Macroglobulinemia, Waldenstrom; Malignant Fibrous Histiocytoma of Bone and Osteosarcoma; Medulloblastoma, Childhood; Medulloepithelioma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndromes, Childhood; Multiple Myeloma/Plasma Cell Neoplasm; Mycosis Fungoides; Myelodysplastic Syndromes; Myelodysplastic/Myeloproliferative Neoplasms; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin Lymphoma, Adult; Non-Hodgkin Lymphoma, Childhood; Non-Small Cell Lung Cancer; Oral Cancer, Childhood; Oral Cavity Cancer, Lip and; Oropharyngeal Cancer; Osteosarcoma and Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell Tumors; Papillomatosis, Childhood; Paranasal Sinus and Nasal Cavity Cancer; Parathyroid Cancer; Penile Cancer; Pharyngeal Cancer; Pineal Parenchymal Tumors of Intermediate Differentiation, Childhood; Pineoblastoma and Supratentorial Primitive Neuroectodermal Tumors, Childhood; Pituitary Tumor; Plasma Cell Neoplasm/Multiple Myeloma; Pleuropulmonary Blastoma, Childhood; Pregnancy and Breast Cancer; Primary Central Nervous System (CNS) Lymphoma; Prostate Cancer; Rectal Cancer; Renal Cell (Kidney) Cancer; Renal Pelvis and Ureter, Transitional Cell Cancer; Respiratory Tract Cancer with Chromosome 15 Changes; Retinoblastoma; Rhabdomyosarcoma, Childhood; Salivary Gland Cancer; Salivary Gland Cancer, Childhood; Sarcoma, Ewing Sarcoma Family of Tumors; Sarcoma, Kaposi; Sarcoma, Soft Tissue, Adult; Sarcoma, Soft Tissue, Childhood; Sarcoma, Uterine; Sézary Syndrome; Skin Cancer (Nonmelanoma); Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Cell Carcinoma; Squamous Neck Cancer with Occult Primary, Metastatic; Stomach (Gastric) Cancer; Supratentorial Primitive Neuroectodermal Tumors, Childhood; T-Cell Lymphoma, Cutaneous; Testicular Cancer; Throat Cancer; Thymoma and Thymic Carcinoma; Thyroid Cancer; Thyroid Cancer, Childhood; Transitional Cell Cancer of the Renal Pelvis and Ureter; Trophoblastic Tumor, Gestational; Unknown Primary Site, Carcinoma of, Adult; Unknown Primary Site, Cancer of, Childhood; Unusual Cancers of Childhood; Ureter and Renal Pelvis, Transitional Cell Cancer; Urethral Cancer; Uterine Cancer, Endometrial; Uterine Sarcoma; Vaginal Cancer; Vulvar Cancer; Waldenström Macroglobulinemia; or Wilms Tumor.

### Viral Indications

Augmentation of cell-based therapies can also be useful as a treatment for chronic viral infections. In certain embodiments, an individual with a chronic viral infection is treated using the methods described herein. In certain embodiments, the chronic viral infection include human immunodeficiency virus (HIV), Hepatitis B virus, Hepatitis C virus, Epstein-Barr virus, Herpes simplex I virus, Herpes Simplex II virus, Human Papilloma virus (HPV), or human cytomegalovirus (hCMV).

### Methods of Treating Latent Viral Disease

The HDACi disclosed herein, are useful in methods of treating latent viral disease. While many latent viral diseases, such as HIV or Herpes, can be effectively treated, there remain significant obstacles to “curing” these diseases (e.g., completely ridding the body of virus or allowing an individual to stop taking antiviral treatments). Treatment with a class I HDACi such as nanatinostat can reactivate latent virus from latent viral reservoirs, and allow for treatment with appropriate cell-based therapies or antiviral drugs. This type of method can be referred to as “purging” or “kick and kill”. In certain embodiments, the chronic viral infection “purged” by the method herein comprises human immunodeficiency virus (HIV), Hepatitis B virus, Hepatitis C virus, Epstein-Barr virus, Herpes simplex I virus, Herpes Simplex II virus, or human cytomegalovirus (hCMV).

The methods and HDACi disclosed herein can be utilized in a method of treating human immunodeficiency virus (HIV). In certain embodiments, the methods and HDACi are useful to reactivate latent viral reservoirs to allow for elimination of the virus. In certain embodiments, the HDACi are administered to an individual to reactivate latent virus followed by treatment with one or more HIV anti-retroviral drugs, immunotherapies, cell based immunotherapies, therapeutic vaccines, or a combination thereof. In certain embodiments, the HDACi comprises, consists essentially, or consists of nanatinostat.

An individual who is HIV positive can be treated with an HDACi, such as nanatinostat to reactivate latent HIV infection for “purging” by subsequent antiviral treatment. The individual can be previously treated with an antiviral regimen. In certain embodiments, the individual has an undetectable viral load by a standard laboratory test such as polymerase chain reaction (PCR). In certain embodiments, the individual has a viral load below 1000 copies/mL. In certain embodiments, the individual has a viral load below 500 copies/mL. In certain embodiments, the individual has a viral load below 200 copies/mL. In certain embodiments, the individual has a viral load below 100 copies/mL. In certain embodiments, the individual has a viral load below 50 copies/mL. In certain embodiments, the individual has a viral load below 1000, 500, 200, 100 or 50 copies/mL for at least 3 months, 6 months or a year before treatment with a latency reversing agent such as an HDACi.

For viral “purging” applications a patient can be pre-treated with an effective amount of a class I HDAC inhibitor before being administered a treatment designed to eliminate the latent virus. In some embodiments, the class I HDACi is nanatinostat. In certain embodiments, nanatinostat administered at a dose of 40 mg/day. In some embodiments, nanatinostat is administered at a dose of about 1 mg/day, about 2 mg/day, about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 60 mg/day, about 70 mg/day, about 80 mg/day, about 90 mg/day, or about 100 mg/day. In certain embodiments, Nanatinostat is administered at a dose of less than 1 mg/day, less than 2 mg/day, less than 5 mg/day, less than 10 mg/day, less than 15 mg/day, less than 20 mg/day, less than 25 mg/day, less than 30 mg/day, less than 35 mg/day, less than 40 mg/day, less than 45 mg/day, less than 50 mg/day, less than 60 mg/day, less than 70 mg/day, less than 80 mg/day, less than 90 mg/day, or less than 100 mg/day. In some embodiments, nanatinostat is administered at a dose of more than 1 mg/day, more than 2 mg/day, more than 5 mg/day, more than 10 mg/day, more than 15 mg/day, more than 20 mg/day, more than 25 mg/day, more than 30 mg/day, more than 35 mg/day, more than 40 mg/day, more than 45 mg/day, more than 50 mg/day, more than 60 mg/day, more than 70 mg/day, more than 80 mg/day, more than 90 mg/day, or more than 100 mg/day. In certain embodiments, nanatinostat is administered at a dose of more than 30 mg/day and less than 50 mg/day. In some embodiments, nanatinostat is administered at a dose of more than 5 mg/day and less than 80 mg/day. In some embodiments, nanatinostat is administered at a dose of more than 10 mg/day and less than 80 mg/day. In some embodiments, nanatinostat is administered at a dose of more than 20 mg/day and less than 80 mg/day. In some embodiments, nanatinostat is administered at a dose of about 1 mg/day, about 2 mg/day, about 5 mg/day, about 6 mg/day, about 7 mg/day, about 8 mg/day, about 9 mg/day, about 10 mg/day, about 11 mg/day, about 12 mg/day, about 13 mg/day, about 14 mg/day, about 15 mg/day, about 16 mg/day, about 17 mg/day, about 18 mg/day, about 19 mg/day, about 20 mg/day, about 22 mg/day, about 23 mg/day, about 25 mg/day, about 27 mg/day, about 28 mg/day, about 30 mg/day, about 32 mg/day, about 33 mg/day, about 35 mg/day, about 40 mg/day, about 45 mg/day, about 50 mg/day, about 60 mg/day, about 70 mg/day, about 80 mg/day, about 90 mg/day, or about 100 mg/day. In certain embodiments, nanatinostat is administered once a day (q.d.), twice a day (b.i.d.), or thrice a day (t.i.d.). In some embodiments, nanatinostat is administered daily, once a week, twice a week, three times a week, four times a week, or five times a week. Nanatinostat can be administered for at least 1, 2, 3, or 4 weeks prior to administering a treatment designed to eliminate the latent virus. Nanatinostat can be administered for at least 1, 2, 3, or 4 months prior to administering a treatment designed to eliminate the latent virus.

In certain embodiments, treatment with nanatinostat is followed by or administered concurrently with an anti-retroviral drug. In certain embodiments, the anti-retroviral drug comprises or consists of Abacavir (Ziagen), Atazanavir (Reyataz), Darunavir (Prezista), Dolutegravir (Tivicay), Efavirenz (Sustiva), Elvitegravir, Emtricitabine (Emtriva), Etravirine (Intelence), Fosamprenavir (Telzir, Lexiva), Lamivudine (Epivir), Lopinavir/ritonavir (Kaletra), Maraviroc (Celsentri), Nevirapine (Viramune), Raltegravir (Isentress), Rilpivirine (Edurant), Ritonavir (Norvir), Tenofovir (Viread), Zidovudine (AZT, Retrovir) and combinations thereof. In certain embodiments, the antiretroviral drug is a combination treatment comprising or consisting of, for example, Efavirenz/Emtricitabine/Tenofovir disoproxil fumarate (Atripla), Atazanavir/Cobicistat (Evotaz), Emtricitabine/Tenofovir (Descovy), Darunavir/Cobicistat (Rezolsta), Elvitegravir/Cobicistat/Emtricitabine/Tenofovir (Stribild), Abacavir/Dolutegravir/Lamivudine (Triumeq), Emtricitabine/rilpivirine/Tenofovir (Odefsey), Rilpivirine/Emtricitabine/Tenofovir (Eviplera), Abacavir/Lamivudine (Kivexa), and Elvitegravir/Cobicistat/Emtricitabine/Tenofovir (Genvoya). Any of these anti-retroviral drugs can be administered in dosage amounts and at times standard for the given anti-retroviral.

In certain embodiments, treatment with nanatinostat to reactivate latent virus is followed by or administered concurrently with a treatment with an immunotherapy. In certain embodiments, the immunotherapy is an antibody or mixture of antibodies that bind an HIV polypeptide. In certain embodiments, the immunotherapy is a cell-based therapy that comprises a CAR T cell, or population thereof, transgenic for a CAR specific for an HIV derived polypeptide, a T cell or population thereof transgenic for a T cell receptor specific for an HIV polypeptide bond to an MHC class I or MHC class II molecule, or a cytotoxic T cell population (CD8+) that specifically lyses HIV infected cells. In certain embodiments, the cell based therapy can be an autologous T-cell population, treated with HDACi to reverse exhaustion or otherwise augment functionality. In certain embodiments, the cell-based therapy has been treated with an HDACi in vitro to augment the cell-based therapy as described above. In certain embodiments, the HDACi that is used to augment the cell-based immunotherapy is nanatinostat, and is applied to the cell-based therapy before administration to a patient treated with an HDACi to reactivate latent virus.

For in vitro applications (e.g., administration in cell culture) a cell-based immunotherapy can be treated with an effective amount of a class I HDACi. In some embodiments, the class I HDACi is Nanatinostat (also referred to as VRx-3996 or CHR-3996). The chemical formula of Nanatinostat is (2-(6-{[(6-Fluoroquinolin-2-yl)methyl]amino}-3-azabicyclo[3.1.0]hex-3-yl)-N-hydroxypyrimidine-5-carboxamide). Nanatinostat is a selective Class I HDAC inhibitor and is disclosed in U.S. Pat. No. 7,932,246, which is incorporated by reference herein in its entirety. An effective amount is one that results in increased histone acetylation in a cell-based immunotherapeutic. In a certain embodiment, the histone with increased acetylation comprises Histone H3. In a certain embodiment, the histone with increased acetylation comprises Histone H3 and the increased acetylation is at lysine 9. In certain embodiments, the cell-based immunotherapy is treated with a concentration of nanatinostat less than about 10 μM. In certain embodiments, the cell-based immunotherapy is treated with a concentration of nanatinostat less than about 5 μM. In certain embodiments, the cell-based immunotherapy is treated with a concentration of nanatinostat less than about 2 μM. In certain embodiments, the cell-based immunotherapy is treated with a concentration of nanatinostat less than about 1 μM. In certain embodiments, the cell-based immunotherapy is treated with a concentration of nanatinostat less than about 900 nM. In certain embodiments, the cell-based immunotherapy is treated with a concentration of nanatinostat less than about 800 nM. In certain embodiments, the cell-based immunotherapy is treated with a concentration of nanatinostat less than about 700 nM. In certain embodiments, the cell-based immunotherapy is treated with a concentration of nanatinostat less than about 600 nM. In certain embodiments, the cell-based immunotherapy is treated with a concentration of nanatinostat less than about 500 nM. In certain embodiments, the cell-based immunotherapy is treated with a concentration of nanatinostat less than about 400 nM. In certain embodiments, the cell-based immunotherapy is treated with a concentration of nanatinostat less than about 300 nM. In certain embodiments, the cell-based immunotherapy is treated with a concentration of nanatinostat less than about 200 nM. In certain embodiments, the cell-based immunotherapy is treated with a concentration of nanatinostat less than about 100 nM. In certain embodiments, the cell-based immunotherapy is treated with a concentration of nanatinostat less than about 50 nM. In certain embodiments, the cell-based immunotherapy is treated with a concentration of nanatinostat greater than about 1 nM. In certain embodiments, the cell-based immunotherapy is treated with a concentration of nanatinostat greater than about 2 nM. In certain embodiments, the cell-based immunotherapy is treated with a concentration of nanatinostat greater than about 5 nM. In certain embodiments, the cell-based immunotherapy is treated with a concentration of nanatinostat greater than about 10 nM. In certain embodiments, the cell-based immunotherapy is treated with a concentration of nanatinostat greater than about 100 nM. In certain embodiments, the nanatinostat is administered between about 1 nM and about 5 μM, between about 1 nM and about 2 μM, between about 1 nM and about 1 μM, between about 1 nM and about 900 nM, between about 1 nM and about 800 nM, between about 1 nM and about 700 nM, between about 1 nM and about 600 nM, between about 1 nM and about 500 nM, between about 1 nM and about 400 nM, between about 1 nM and about 300 nM, between about 1 nM and about 200 nM, between about 1 nM and about 100 nM, between about 1 nM and about 50 nM, between about 1 nM and about 25 nM, between about 10 nM and about 5 μM, between about 10 nM and about 2 μM, between about 10 nM and about 1 μM, between about 10 nM and about 900 nM, between about 10 nM and about 800 nM, between about 10 nM and about 700 nM, between about 10 nM and about 600 nM, between about 10 nM and about 500 nM, between about 10 nM and about 400 nM, between about 10 nM and about 300 nM, between about 10 nM and about 200 nM, between about 1 nM and about 100 nM, between about 10 nM and about 50 nM, between about 10 nM and about 25 nM. Nanatinostat can be incubated with a cell-based immunotherapy for about 1, 2, 4, 8, 16, 24, or 48 hours. Nanatinostat can be incubated with a cell-based immunotherapy for at least about 1, 2, 4, 8, 16, 24, or 48 hours. Nanatinostat can be incubated with a cell-based immunotherapy for no more than about 1, 2, 4, 8, 16, 24, or 48 hours.

In certain embodiments, an HDACi that is not nanatinostat can be combined with nanatinostat in a method of purging latent viral reservoir. In certain embodiments, the HDACi that is not nanatinostat is used to reactivate latent virus, and nanatinostat is used to augment a cell-based immunotherapeutic that targets the virus. In certain embodiments, nanatinostat is used to reactivate latent virus, and the HDACi that is not nanatinostat is used to augment a cell-based immunotherapeutic that targets the virus. In certain embossments, the virus is HIV. In certain embodiments, the HDACi that is not nanatinostat comprises quisinostat (JNJ-26481585 (N-hydroxy-2-(4-((((1-methyl-1H-indol-3-yl)methyl)amino)methyl)piperidin-1-yl)pyrimidine-5-carboxamide)), R306465/JNJ-16241199 (N-hydroxy-5-(4-(naphthalen-2-ylsulfonyl)piperazin-1-yl)pyrimidine-2-carboxamide), Belinostat/PXD101, trichostatin A/TSA (7-[4-(dimethylamino)phenyl]-N-hydroxy-4,6-dimethyl-7-oxohepta-2,4-dienamide), ITF2357, CBHA, Givinostat/ITF2357, romidepsin, PCI-24781, depsipeptide (FR901228 or FK228), butyrate, phenylbutyrate, valproic acid, AN-9, CI-994, Entinostat/MS-275/SNDX-275, mocetinostat/MGCD0103 (N-(2-aminophenyl)-4-((4-pyridin-3-ylpyrimidin-2-ylamino)methyl)benzamide), m-carboxycinnamic acid, bishydroxamic acid, suberic bishydroxamic acid, oxamflatin, ABHA, SB-55629, pyroxamide, propenamides, aroyl pyrrolyl hydroxamides, or LAQ824 (((E)-N-hydroxy-3-[4-[[2-hydroxyethyl-[2-(1H-indol-3-yl)ethyl]amino]methyl]phenyl]prop-2-enamide), panobinostat/LBH-589, vorinsotat/SAHA, chidamide, or 4SC-202.

Described herein, in specific numbered embodiments below are:

1. A method of treating human immunodeficiency (HIV) infection in an individual comprising: administering to an individual with an HIV infection an effective amount of a histone deacetylase inhibitor(HDACi), wherein the HDACi comprises nanatinostat, quisinostat (JNJ-26481585 (N-hydroxy-2-(4-((((1-methyl-1H-indol-3-yl)methyl)amino)methyl)piperidin-1-yl)pyrimidine-5-carboxamide)), R306465/JNJ-16241199 (N-hydroxy-5-(4-(naphthalen-2-ylsulfonyl)piperazin-1-yl)pyrimidine-2-carboxamide), Belinostat/PXD101, trichostatin A/TSA (7-[4-(dimethylamino)phenyl]-N-hydroxy-4,6-dimethyl-7-oxohepta-2,4-dienamide), ITF2357, CBHA, Givinostat/ITF2357, romidepsin, PCI-24781, depsipeptide (FR901228 or FK228), butyrate, phenylbutyrate, valproic acid, AN-9, CI-994, Entinostat/MS-275/SNDX-275, mocetinostat/MGCD0103 (N-(2-aminophenyl)-4-((4-pyridin-3-ylpyrimidin-2-ylamino)methyl)benzamide), m-carboxycinnamic acid, bishydroxamic acid, suberic bishydroxamic acid, oxamflatin, ABHA, SB-55629, pyroxamide, propenamides, aroyl pyrrolyl hydroxamides, or LAQ824 (((E)-N-hydroxy-3-[4-[[2-hydroxyethyl-[2-(1H-indol-3-yl)ethyl]amino]methyl]phenyl]prop-2-enamide), panobinostat/LBH-589, vorinsotat/SAHA, chidamide, or 4SC-202, wherein the individual with an HIV infection has an HIV viral load of less than 1000 copies of HIV RNA per milliliter of blood.
2. The method of embodiment 1, wherein the individual has an HIV viral load of less than 100 copies of HIV RNA per milliliter.
3. The method of embodiment 1 or 2, wherein the HDACi is administered at a dose of less than 80 mg per day.
4. The method of embodiment 1 or 2, wherein the HDACi is administered at a dose of less than 40 mg per day.
5. The method of embodiment 1 or 2, wherein the HDACi is administered at a dose of less than 20 mg per day.
6. The method of any one of embodiments 1 to 5, further comprising administering an anti-HIV treatment to the individual with an HIV infection.
7. The method of embodiment 6, wherein the anti-HIV treatment comprises an anti-retroviral drug or pharmaceutically acceptable salt thereof.
8. The method of embodiment 7, wherein the anti-retroviral drug or pharmaceutically acceptable salt thereof is selected form the list consisting of Abacavir, Atazanavir, Darunavir, Dolutegravir, Efavirenz, Elvitegravir, Emtricitabine, Etravirine, Fosamprenavir, Lamivudine, Lopinavir, Maraviroc, Nevirapine, Raltegravir, Rilpivirine, Ritonavir, Tenofovir, Zidovudine, and combinations thereof.
9. The method of embodiment 6, wherein the anti-HIV treatment comprises an immunotherapy.
10. The method of embodiment 9 wherein the immunotherapy comprises an antibody that binds to an HIV derived polypeptide.
11. The method of embodiment 9, wherein the immunotherapy comprises a T-cell population.
12. The method of embodiment 11, wherein the T-cell population is transgenic for a chimeric antigen receptor specific for an HIV derived polypeptide.
13. The method of embodiment 11, wherein the T-cell population is a cytotoxic T cell population that specifically lyses HIV infected cells.
14. The method of embodiment 9, wherein the immunotherapy comprises a natural killer cell population.
15. The method of embodiment 14, wherein the natural killer cell population is transgenic for a chimeric antigen receptor specific for an HIV derived polypeptide.
16. The method of any one of embodiments 9 to 15 wherein the immunotherapy is contacted with nanatinostat in vitro prior to administration to the individual with an HIV infection.
17. The method of embodiment 16, wherein the concentration of nanatinostat is an amount sufficient to increase acetylation of histone H3.
18. The method of embodiment 17, wherein the concentration of nanatinostat is less than about 1 micromolar.
19. The method of embodiment 17, wherein nanatinostat is contacted with the immunotherapy for at least 2 hours.
20. The method of embodiment 17, wherein nanatinostat is contacted with the immunotherapy for at least 16 hours.
21. The method of anyone of embodiments 1 to 20, wherein the individual with an HIV infection has previously received an anti-HIV treatment.
22. The method of any one of embodiments 1 to 20, wherein the anti-HIV treatment is an anti-retroviral drug or pharmaceutically acceptable salt thereof.

### Non-HDACi Latency Reversing Agents

Additionally, non-HDACi viral latency reversing agents can be employed in combination with an HDACi in a step to reactivate latent virus either before or during treatment with a n immunotherapeutic or an antiviral drug. In certain embodiments, the non-HDACi viral latency reversing agent comprises or consists essentially of protein kinase C (PKC) modulator such as bryostatin-1 or an analog thereof. In certain embodiments, the non-HDACi viral latency reversing agent comprises or consists essentially of interleukin-7 (IL-7), IL-7 agonists, such as, raltegravir or maraviroc. In certain embodiments, the non-HDACi viral latency reversing agent comprises or consists essentially of interleukin-15 (IL-15) or IL-15 agonists. In certain embodiments, the non-HDACi viral latency reversing agent comprises or consists essentially of disulfram. In certain embodiments, the non-HDACi viral latency reversing agent comprises or consists essentially of a Toll-like receptor agonist, such as, MGN1703. In certain embodiments, the non-HDACi viral latency reversing agent comprises or consists essentially of Ingenol-B. In certain embodiments, the non-HDACi viral latency reversing agent comprises or consists essentially of a Bromodomain and Extraterminal inhibitor (BETi), such as, JQ1, I-BET, or I-BET151.

### Vaccine Adjuvant

Since HDACi can improve functional aspects of components of the cellular immune system, as shown herein, such as T cells and NK cells, HDACi can serve as a vaccine adjuvant. In certain embodiments, an HDACi can serve as an adjuvant to be included in a formulation comprising the HDACi and a prophylactic vaccine (e.g., a vaccine administered prior to infection with a bacteria or virus intended to prevent infection or symptoms). The HDACi can be included as an adjuvant in a prophylactic vaccine that is administered subcutaneously, intramuscularly, orally or intravenously. The HDACi can be included along with other common adjuvants such as alum or squalene oil, or any other adjuvant suitable in creating local inflammation at the site of an injection. In certain embodiments, the HDACi comprises or consists essentially of quisinostat (JNJ-26481585 (N-hydroxy-2-(4-((((1-methyl-1H-indol-3-yl)methyl)amino)methyl)piperidin-1-yl)pyrimidine-5-carboxamide)), R306465/JNJ-16241199 (N-hydroxy-5-(4-(naphthalen-2-ylsulfonyl)piperazin-1-yl)pyrimidine-2-carboxamide), Belinostat/PXD101, trichostatin A/TSA (7-[4-(dimethylamino)phenyl]-N-hydroxy-4,6-dimethyl-7-oxohepta-2,4-dienamide), ITF2357, CBHA, Givinostat/ITF2357, romidepsin, PCI-24781, depsipeptide (FR901228 or FK228), butyrate, phenylbutyrate, valproic acid, AN-9, CI-994, Entinostat/MS-275/SNDX-275, mocetinostat/MGCD0103 (N-(2-aminophenyl)-4-((4-pyridin-3-ylpyrimidin-2-ylamino)methyl)benzamide), m-carboxycinnamic acid, bishydroxamic acid, suberic bishydroxamic acid, oxamflatin, ABHA, SB-55629, pyroxamide, propenamides, aroyl pyrrolyl hydroxamides, or LAQ824 (((E)-N-hydroxy-3-[4-[[2-hydroxyethyl-[2-(1H-indol-3-yl)ethyl]amino]methyl]phenyl]prop-2-enamide), panobinostat/LBH-589, vorinsotat/SAHA, chidamide, nanatinostat, or 4SC-202. In certain embodiments, the HDACi comprises or consists essentially of nanatinostat.

In certain embodiments, the HDACi is included in a formulation at a concentration of at least about 1 mg/mL, about 2 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 150 mg/mL, 200 mg/mL, 300 mg/mL, 400 mg/mL, or about 500 mg/mL. In certain embodiments, the HDACi is included in a vaccine composition at a concentration of about 1 mg/mL, about 2 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 150 mg/mL, 200 mg/mL, 300 mg/mL, 400 mg/mL, or about 500 mg/mL. In certain embodiments, the HDACi is included in a vaccine composition at a concentration no more than about 1 mg/mL, about 2 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 150 mg/mL, 200 mg/mL, 300 mg/mL, 400 mg/mL, or about 500 mg/mL.

In certain embodiments, nanatinostat is included in a formulation at a concentration of at least about 1 mg/mL, about 2 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 150 mg/mL, 200 mg/mL, 300 mg/mL, 400 mg/mL, or about 500 mg/mL. In certain embodiments, nanatinostat is included in a vaccine composition at a concentration of about 1 mg/mL, about 2 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 150 mg/mL, 200 mg/mL, 300 mg/mL, 400 mg/mL, or about 500 mg/mL. In certain embodiments, nanatinostat is included in a vaccine composition at a concentration of nom more than about 1 mg/mL, about 2 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 150 mg/mL, 200 mg/mL, 300 mg/mL, 400 mg/mL, or about 500 mg/mL.

### EXAMPLES

### Example 1-Treatment of Splenocytes with Nanatinostat Reduces FoxP3+ Regulatory T Cells

Nanatinostat specifically reduces CD4+, CD25+, and FoxP3+ regulatory cells. This reduction is particularly striking when compared to another HDAC inhibitor entinostat.

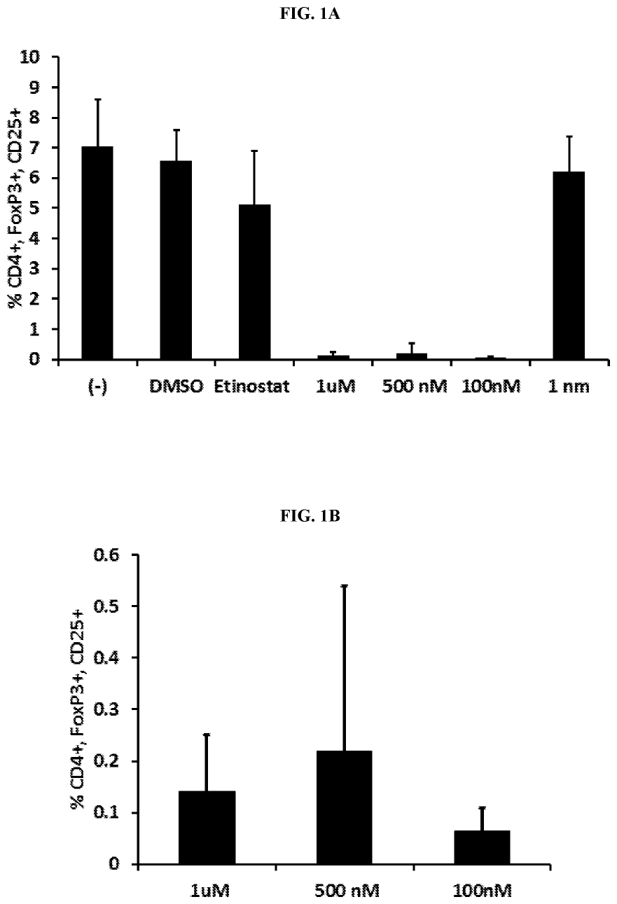

For this experiment 7 treatment groups were created (untreated, DMSO treated, entinostat at 1 μM, nanatinostat at 1 μM, nanatinostat at 500 nM, nanatinostat at 100 nM, and nanatinostat at 1 nM. Each treatment was performed on splenocytes isolated from 5 naïve BALB/c mice (n=5 per treatment group). Spleens were processed in to single cell suspension using Miltenyi Gentlemacs. Red blood cells were lysed using RBC lysis buffer. Cells were counted and re-suspended in RPMI media containing 10% FBS. Cells from each spleen were seeded in a 12 well plate according to the table below at 3×10⁶ cells/well. Test agents were added to each plate at the concentrations indicated in the table below and were incubated for 24 hours prior to FACS analysis. FACS analysis consisted of the following markers: live/dead, CD45, CD3, CD4, CD25 and FoxP3. Results are shown below in Table 1. Compared to entinostat at 1 μM, nanatinostat led to nearly 47-fold reduction in FoxP3, CD25+ T cells (last column of Table 1 and FIG. 1A). This suppressive effect was seen at least as low as 100 nM (last column of Table 1 and FIG. 1B).

TABLE 1
nanatinostat reduces FoxP3+, CD25+ T regulatory cells. Shown are percentages of the indicated cell population ± SD

| | | | | | | | CD45+ | CD45+ | CD45+ |
|---|---|---|---|---|---|---|---|---|---|
| | | | | | CD45+ | CD45+ | CD3+ | CD3+ | CD3+ |
| | | | | CD45+ | CD3+ | CD3+ | CD4+ | CD4+ | CD4+ |
| | | | CD45+ | CD3+ | CD4+ | CD4+ | FoxP3+, | FoxP3− | FoxP3+, |
| group | Live | CD45+ | CD3+ | CD4+ | CD25+ | FoxP3+ | CD25− | CD25+ | CD25+ |
| untreated | 98.3 ± 0.35 | 97.5 ± 1.0 | 42.6 ± 1.79 | 29.7 ± 1.32 | 8.26 ± 1.63 | 13 ± 1.41 | 5.13 ± 0.18 | 0.69 ± 0.15 | 7.05 ± 1.55 |
| DMSO | 98.2 ± 0.37 | 97.6 ± 0.57 | 43.1 ± 1.69 | 29.8 ± 1.48 | 7.74 ± 1.1 | 13.5 ± 1.63 | 5.56 ± 0.63 | 0.66 ± 0.17 | 6.58 ± 1.02 |
| Entinostat | 97.6 ± 0.33 | 99.1 ± 0.27 | 53.9 ± 2.76 | 38.5 ± 2.33 | 10.4 ± 2.09 | 10.1 ± 2.82 | 2.33 ± 0.83 | 4.57 ± 0.89 | 5.12 ± 1.77 |
| 1 uM | | | | | | | | | |
| Nanatinostat | 84.7 ± 2.75 | 99.6 ± 0.17 | 94.7 ± 0.98 | 82.2 ± 1.4 | 3.57 ± 1.76 | 3.85 ± 1.76 | 0.8 ± 0.44 | 1.96 ± 0.48 | 0.14 ± 0.11 |
| 1 uM | | | | | | | | | |
| Nanatinostat | 87.2 ± 1.88 | 99.5 ± 0.29 | 94 ± 4.4 | 80.6 ± 5.32 | 3.03 ± 1.35 | 2.36 ± 1.35 | 0.47 ± 0.35 | 1.69 ± 0.36 | 0.22 ± 0.32 |
| 500 nM | | | | | | | | | |
| Nanatinostat | 91.4 ± 2.47 | 99.6 ± 0.12 | 94 ± 0.83 | 75.3 ± 2.13 | 1.89 ± 1.13 | 1.62 ± 1.13 | 0.34 ± 0.27 | 1.22 ± 0.24 | 0.064 ± 0.044 |
| 100 nM | | | | | | | | | |
| Nanatinostat | 98.4 ± 0.36 | 98.6 ± 0.36 | 46.9 ± 1.87 | 32.4 ± 1.47 | 7.93 ± 1.51 | 12 ± 1.51 | 4.47 ± 0.68 | 1.14 ± 0.31 | 6.2 ± 1.18 |
| nM | | | | | | | | | |

### Example 2—Nanatinostat Increases Efficacy of Immunotherapy in a Mouse Xenograft Model

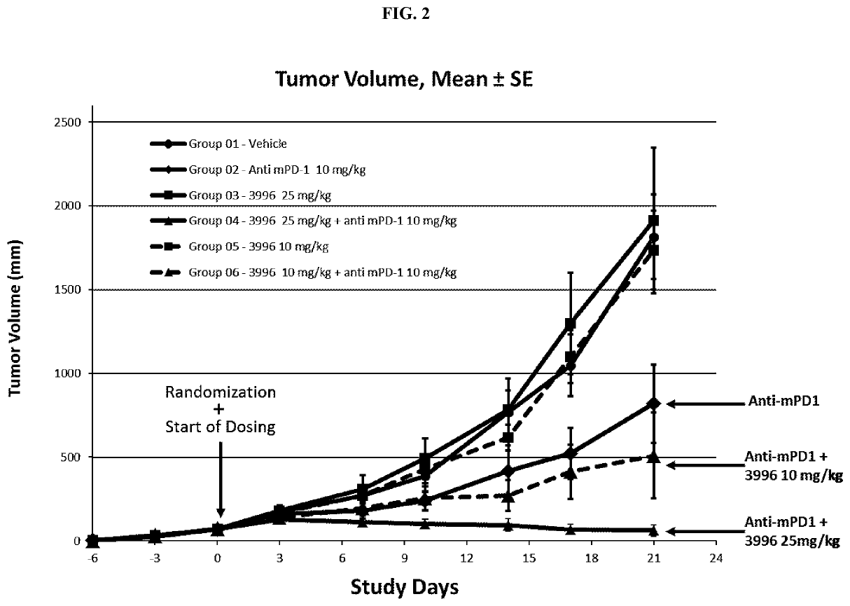

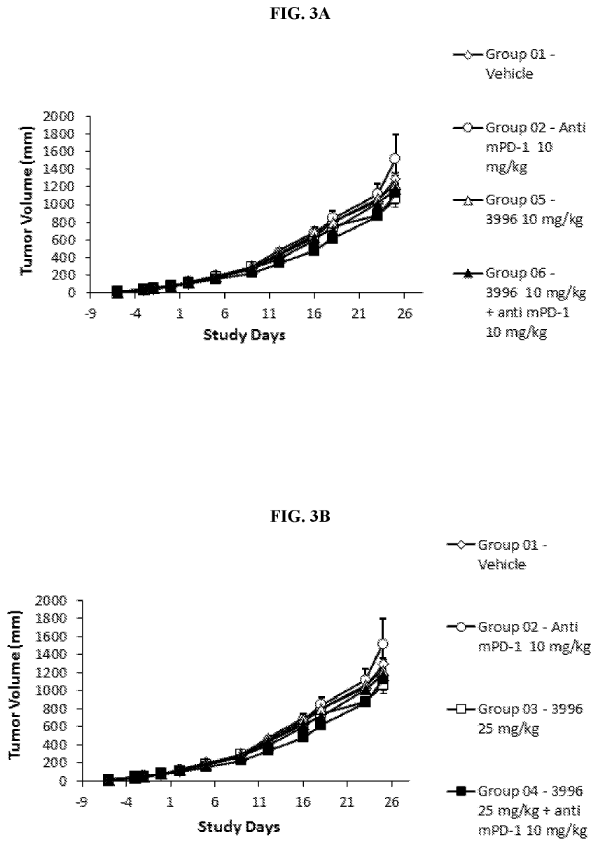

The effect of nanatinostat (CHR-3996) seen on regulatory T cells could lead to an enhancement of the efficacy of immunotherapeutics such as those that target the PD-1/PD-L1 axis. In this example, nanatinostat treatment was combined with anti-PD-1 antibody treatment in two different tumor xenograft models 4T1 and CT26. Each model was tested in 6 different treatment groups (vehicle, anti-PD-1 at 10 mg/kg, nanatinostat at 25 mg/kg, nanatinostat at 10 mg/kg, anti-PD-1 at 10 mg/kg with nanatinostat at 25 mg/kg, anti-PD-1 at 10 mg/kg with nanatinostat at 10 mg/kg), each group consisted of 8 animals. Animals were inoculated in the right rear flank with either 4T1 or CT26, dosing was started when tumors were 65-90 mm³ and continued for 21 days. Animals were dosed daily with nanatinostat and twice weekly with anti-PD-1. FIG. 2 shows that mice receiving CT26 tumor exhibited greater reduction in tumor growth with a combination of anti-PD-1 and nanatinostat (filled shapes) compared with either PD-1 or nanatinostat alone. This was seen for both concentrations of nanatinostat 10 mg/kg/day and 25 mg/kg/day. FIG. 3A and FIG. 3B show that the 4T1 tumor line was resistant to this effect. Indeed this tumor was resistant to anti-PD-1 treatment alone, indicating that HDAC treatment with nanatinostat can specifically synergize with immunotherapies such as anti-PD-1 and potentially all checkpoint inhibitors.

### Example 3—Nanatinostat Increases T Cell Tumor Infiltration in a Mouse Xenograft Model

Nanatinostat alone and in combination with anti-mPD-1 was evaluated in the CT26 subcutaneous tumor model in Balb/c mice. Animals were dosed orally with nanatinostat at 25 mg/kg daily, and intraperitoneally with anti-mPD-1 at 10 mg/kg on a bi-weekly schedule. 8 animals per group were selected and tumors were collected for FACS and qPCR analysis on Day 9, 12-13 hours post dose. The remaining animals continued to receive their respective treatments until Day 21. Nanatinostat was tolerated well. As shown in Table 2 at day 9, the combination treatment group (nanatinostat and anti PD-1) induced the highest tumor growth inhibition (57%), and the anti PD-1 only and nanatinostat only groups induced partial tumor growth inhibition of 36% and 33% respectively.

TABLE 2
inhibition of tumor growth

| | Day 3 | Day 4 | Day 7 | Day 9 |
|---|---|---|---|---|
| Group 01 - Vehicle | | | | |
| for Nanatinostat | | | | |
| Group 02 - Anti | 16.69% | 33.06% | 36.86% | 36.06% |
| mPD-1 10 mg/kg | | | | |
| Group 03 - | 16.20% | 23.31% | 38.94% | 33.81% |
| Nanatinostat | | | | |
| 25 mg/kg | | | | |
| Group 04 - | 24.37% | 32.93% | 49.88% | 57.29% |
| Nanatinostat | | | | |
| 25 mg/kg + anti | | | | |
| mPD-1 10 mg/kg | | | | |

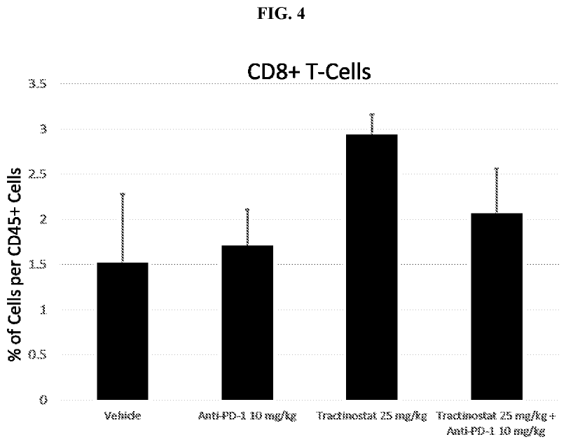

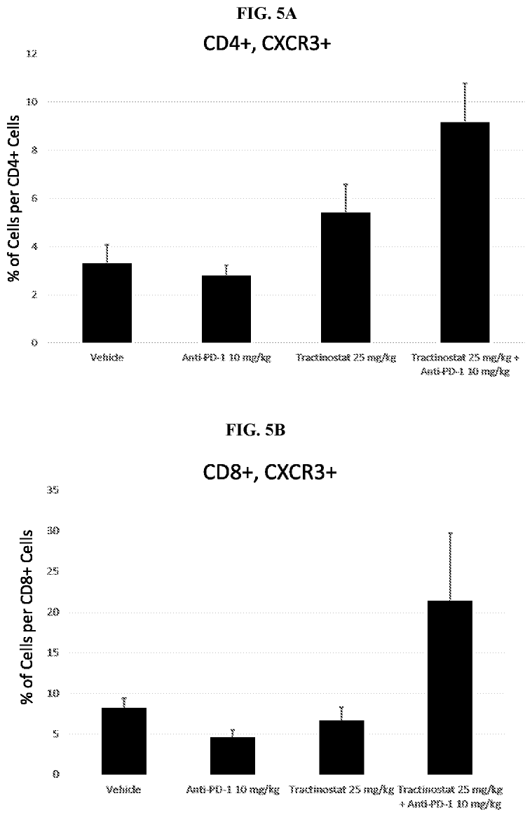

As shown in FIG. 4, T cell infiltration significantly increased in the Nanatinostat treated group compared to the vehicle or anti-PD-1 group (p-value 0.007 for both). Specifically, CD8+ T-cell population was higher in the nanatinostat treated group compared to the vehicle group, while CD4+ population and T regulatory cell population were not significantly different across groups (not shown). As shown in FIGS. 5A and 5B, the CXCR3 expressing cell population was significantly higher in groups treated with nanatinostat+ anti-PD-1 compared to the group only treated with anti PD-1 in CD4+ (FIG. 5A; p-value versus vehicle 0.015, p-value versus anti-PD-1 0.07), and CD8+ T cells (FIG. 5B; p-value versus anti-PD-1 0.0.21), while no significant difference was observed when only treated with nanatinostat compared to the vehicle group.

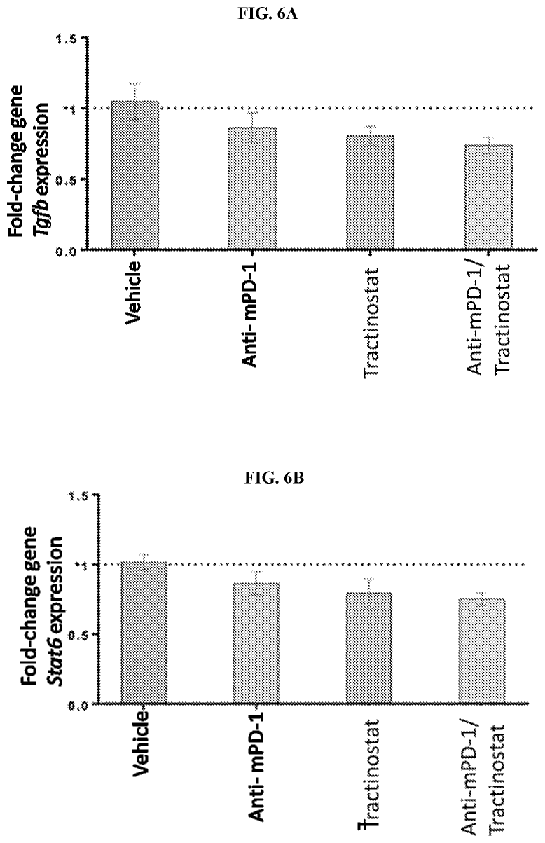

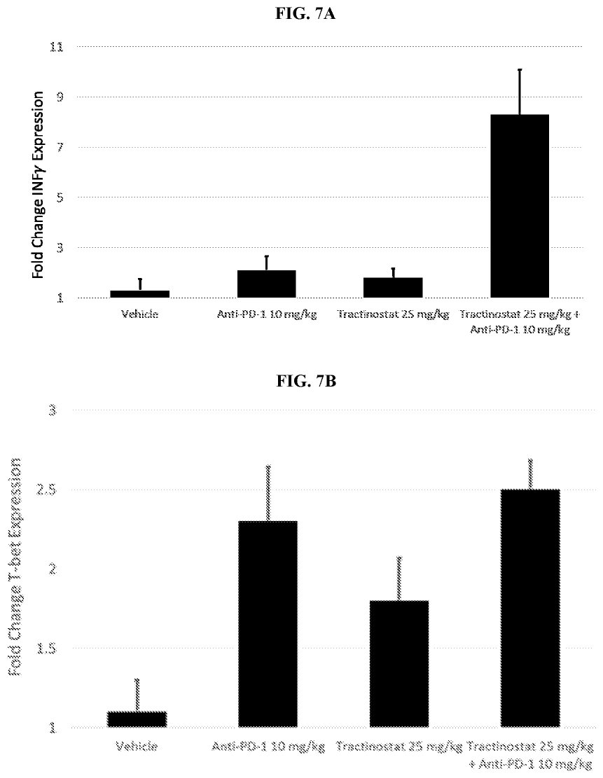

The fold change in gene expression (relative to the vehicle control group) of immunosuppressive markers TGFß1 and Stat6 trended to decrease in the treatment groups compared to the control group as shown in FIGS. 6A (TGFβ1) and 6B (Stat6). Conversely, FIG. 7A (IFN-γ), FIG. 7A (Tbet) show that fold change in gene expression were the highest in the combination group.

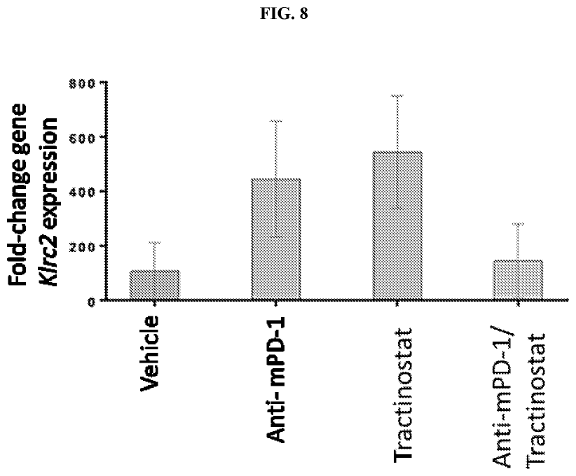

Interestingly, as shown in FIG. 8 both Nanatinostat and PD-1 separately increased KLRC2 expression on NK cells, but this increase was blocked by the combination.

### Methods

Animals:

144 female Balb/c mice (date of birth: Aug. 1, 2017) purchased from Jackson Laboratories were inoculated for the study. Animals were housed for a stabilization period of 5 days prior to inoculation. Animals were housed in individual HEPA ventilated cages (Innocage® IVC, Innovive USA). Fluorescent lighting was provided on a 12-hour cycle. Temperature and humidity was monitored and recorded daily and maintained to the maximum extent possible between 68-74° F. (20-23° C.) and 30-70% humidity, respectively. 2920X.10 18% soy irradiated rodent feed (Envigo) and autoclaved acidified water (pH2.5-3) was provided ad libitum.

Tumor Cell Preparation:

CT26 cells were cultured as per ATCC's recommended culture protocol. For inoculation, cells were washed in PBS, counted, and resuspended in cold PBS at a concentration of 250,000 viable cells/100 μl. Cell suspension was kept on ice during transport to the vivarium. Cells were prepared for injections by withdrawing cells into a chilled 1 ml syringe fitted with a 26G 7/8 (0.5 mm×22 mm) needle.

Tumor Implantation:

Animals were prepared as needed for injection using standard approved anesthesia, and the mice were shaved prior to injection. One mouse at a time was immobilized and the site of injection was disinfected with an alcohol swab. 100 μl of the cell suspension was subcutaneously injected into the rear flank of the mouse. Mice were marked by ear tagging.

Tumor Measurements:

Animals were monitored daily for palpable tumors, or any changes in appearance or behavior. Once tumors were palpable, they were measured using calipers. Tumor volume was calculated using the following equation (longest diameter×shortest diameter²)/2.

FACS Analysis:

On Day 9, N=8/arm were harvested tumors for PD assessment 12-13 hours post last dose. Half of each tumor was placed in transfer buffer for FACS analysis.

QPCR Analysis:

On Day 9, N=8/arm was harvested tumors for PD assessment. Half of each tumor was flash frozen for subsequent qPCR analysis. Fragments of flash frozen tumors were transferred in RLT PLUS buffer (Qiagen) and loaded into labelled and prefilled tubes with beads and homogenized for 60 seconds at maximum speed. RNA was extracted with AllPrep DNA/RNA Mini kit, purchased from Qiagen, according to the manufacturer's protocol. RNA quantity and purity was evaluated by Synergy 2 Multi-Mode Reader according to the Biotec Protocol. 750 ng total RNA from each sample was used to generate cDNA in a 20 μl reaction. The reaction was performed to the standard of Superscript III First-Strand Synthesis protocol by ThermoFisher by using Eppendorf Mastercycler Pro. Following reverse transcription, template RNA was digested by using E. Coli RNAse H, according to the manufacturer's instruction. 37.5 ng cDNA of each sample was used for Gene Expression PCR in a total volume of 10 μl reaction. Each sample was analyzed in triplicates and qRT-PCR was performed with 384-well platform ABI-ViiA7 Fast real-time PCR system using standard parameters suggested by the manufacturer. This study used specific TaqMan Gene Expression Assay purchased from Thermo Fisher. Gene expression data was analyzed on the ViiA7 system using ABI 2.1 software to generate the raw data. Mouse β-actin was used as housekeeping gene.

### Example 4-In Vitro Treatment of Tumor Infiltrating Lymphocytes with Nanatinostat in Autologous Adoptive T Cell Immunotherapy

A patient either diagnosed with, or suspected of having, breast cancer has tumor infiltrating lymphocytes isolated from biopsied tissue. Cells are expanded in X-VIVO medium (Lonza) in the presence of IL-2, anti-CD3, and irradiated feeder cells. Once a sufficient number of cells are generated (at least 1×10⁹) the cells are incubated with 100 nM of nanatinostat for 24 hours. After treatment T cells are harvested and administered to the patient (at least 1×10⁹).

### Example 5—In Vitro Treatment of Tumor Infiltrating Lymphocytes with Nanatinostat in Autologous Adoptive T Cell Immunotherapy in a Patient that has been Pre-Treated with Nanatinostat

This example operates per example 4 except that that the patient has been orally treated with 2 mg of nanatinostat weekly for 4 weeks before isolation of tumor infiltrating lymphocytes.

### Example 6—Reversal of T Cell Exhaustion by Nanatinostat

To test whether nanatinostat can be used to reverse T cell exhaustion, whether by itself or in combination with PD-1, a standard T cell exhaustion assay was used. Peripheral Blood Mononuclear Cells (PBMC) were separated over a density gradient from 3 single human donor buffy coats. PBMC were stimulated with pooled pathogen-specific class I peptides (CEFT) to generate an exhausted T cell population. Cells were then re-stimulated with CEFT and autologous monocyte-derived dendritic cells for a further time before testing in a final re-stimulation assay using replenished DC and CEFT to monitor reversal of exhaustion. The stimulation assays were carried out in the presence of the HDAC inhibitor nanatinostat (Nstat) alone or in combination with anti-PD-1, or in the presence of another class I HDACi entinostat. One dose of HDAC inhibitor was tested and a single dose of anti-PD-1 was used. An unstimulated control and a reference HDAC inhibitor were also plated. Cultures were pulsed with 3H-thymidine and proliferation assessed. Supernatants were also harvested for cytokine analysis by multiplex. Samples of cells were stained with an antibody panel directed against CD4, CD8, PD-1, IL-15R, TIGIT, CD45RO, CCR7, IFNγ and CD25 and characterized using flow cytometry in order to confirm their exhausted phenotype, and whether expression levels were altered following exposure to the combination therapies. PBMC were also phenotyped using the above panel at day 0 to determine the initial frequency of cell populations.

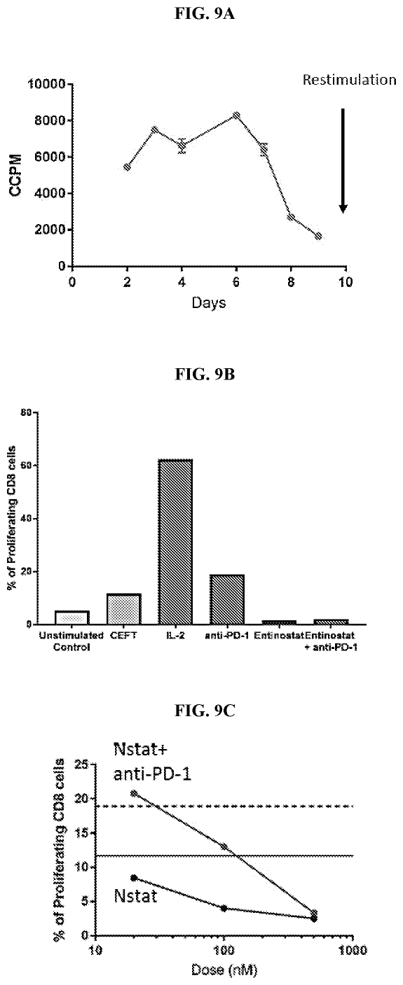

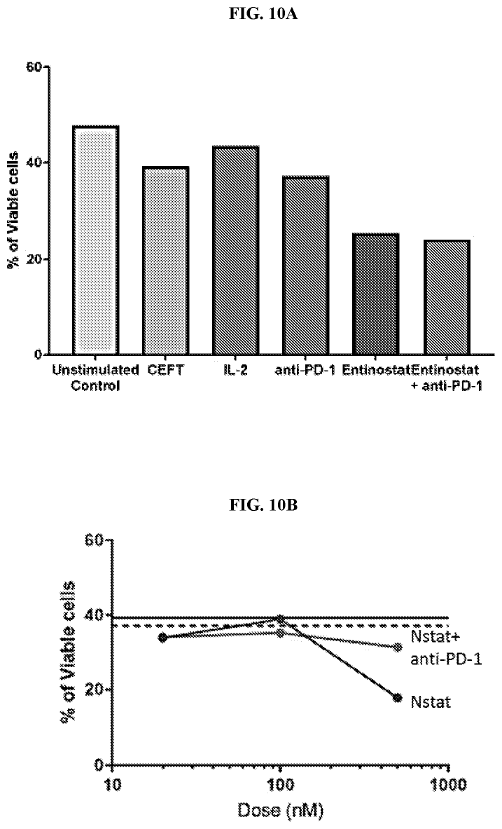

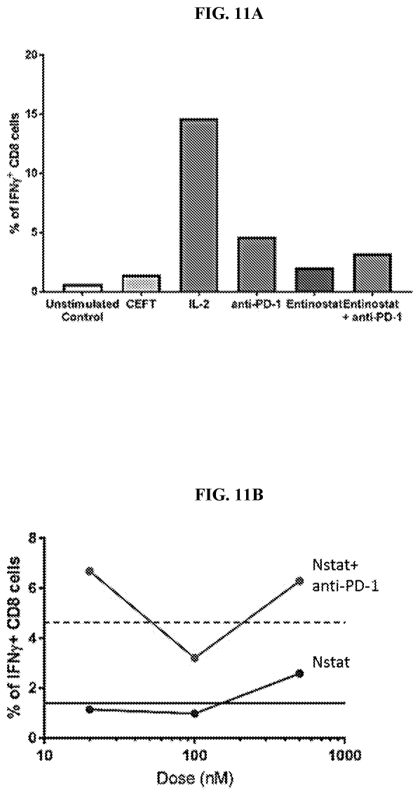

The data show that the T cell exhaustion protocol was effective. FIG. 9A shows that PBMC proliferation peaked at day 6 before rapidly declining by day 10 at which time T cells were restimulated with (CEFT). FIGS. 9B and 9C show that anti-PD-1 and CEFT restored proliferation of CD8+ T cells compared to CEFT alone. Nanatinostat alone had a negative effect on CEFT CD8+ T cell proliferation, however when combined with anti-PD-1 (Pembrolizumab) treatment proliferation was restored at 10 nM and 100 nM. This reduction in proliferation in response to PD-1 was not a result of reduced cell viability as shown in FIG. 10. Overall nanatinostat was well tolerated by the cells compared to the class I HDAC1 and HDAC3 inhibitor Entinostat (compare FIGS. 10A and 10B). As shown in FIG. 11, restimulated CD8+ T cells treated with nanatinostat and PD-1 inhibitor antibody secreted more INF-γ than either PD-1 inhibitor alone or nanatinostat alone (FIG. 11B), while Entinostat actually reduced the amount of IFN-γ released, either alone or in combination with anti-PD-1 (FIG. 11A). Nanatinostat alone had little effect on IFN-γ release by restimulated CD8+ T cells (FIG. 11B).

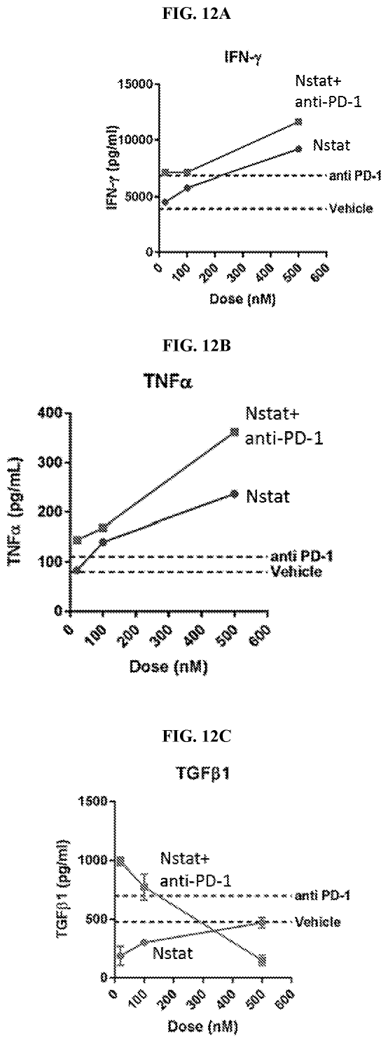

FIG. 12 shows that analysis of cytokines from the supernatant of restimulated CD8+ T cells indicated that the combination of anti-PD1 and nanatinostat increased release of immunostimulatory IFN-γ (FIG. 12A) and TNFα (FIG. 12B), while decreasing the release of immunoinhibitory TGFβ (FIG. 12C).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for augmenting a cell-based immunotherapy comprising contacting a cell-based immunotherapy in vitro with an HDAC inhibitor (HDACi), wherein the HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide).

2. The method of claim 1, wherein the method reverses T cell exhaustion.

3. The method of claim 1, wherein the concentration of the HDACi is an amount sufficient to increase acetylation of histone H3.

4. The method of claim 3, wherein the concentration of the HDACi is less than about 1 micromolar.

5. The method of claim 3, wherein the concentration of the HDACi is at least about 400 nanomolar.

6. The method of any one of claims 1 to 5, wherein the HDACi is contacted with the cell-based immunotherapy for at least 2 hours.

7. The method of any one of claims 1 to 5, wherein the HDACi is contacted with the cell-based immunotherapy for at least 16 hours.

8. The method of any one of claims 1 to 7, wherein the method comprises contacting the cell-based immunotherapy with interleukin-15.

9. The method of claim 8, wherein the interleukin-15 is contacted with the cell-based immunotherapy at a concentration of about 1 to about 100 ng/mL.

10. The method of claim 8, wherein the interleukin-15 is contacted with the cell-based immunotherapy at a concentration of about 5 to about 25 ng/mL.

11. The method of claim 8, wherein the interleukin-15 is contacted with the cell-based immunotherapy at a concentration of about 10 ng/mL.

12. The method of any one of claims 1 to 11, wherein the method comprises contacting the cell-based immunotherapy with a checkpoint inhibitor.

13. The method of claim 12, wherein the checkpoint inhibitor is an antibody that targets PDL-1 or PD-1.

14. The method of any one of claims 1 to 13, wherein the cell-based immunotherapy comprises a T-cell population.

15. The method of claim 14, wherein the T-cell population comprises a primary T-cell population derived from a healthy individual.

16. The method of claim 14, wherein the T-cell population comprises a primary T-cell population derived from an individual afflicted with a disease.

17. The method of any one of claims 14 to 16, wherein the T-cell population further comprises a chimeric antigen receptor (CAR).

18. The method of any one of claims 14 to 16, wherein the method further comprises stimulating the T-cell population with a tumor associated antigen.

19. The method of any one of claims 14 to 16, wherein the method further comprises stimulating the T-cell population with a pro-inflammatory cytokine.

20. The method of any one of claims 14 to 19, wherein the T-cell population is enriched for CD4 positive T cells.

21. The method of any one of claims 14 to 19, wherein the T-cell population is enriched for CD8 positive T cells.

22. The method of any one of claims 14 to 21, wherein FoxP3 expression is reduced in the T-cell population after contacting the cell-based immunotherapy with an HDACi.

23. The method of any one of claims 14 to 21, wherein secretion of interferon gamma is increased in the T-cell population after contacting the cell-based immunotherapy with an HDACi.

24. The method of any one of claims 14 to 21, wherein cell-surface expression of CXCR3 is increased in the T-cell population after contacting the cell-based immunotherapy with an HDACi.

25. The method of any one of claims 1 to 13, wherein the cell-based therapy comprises a T-cell line.

26. The method of claim 25, wherein the T cell line comprises a chimeric antigen receptor.

27. The method of claim 25 or 26, wherein FoxP3 expression is reduced in the T cell line after contacting the cell-based immunotherapy with an HDACi.

28. The method of claim 25 or 26, wherein secretion of interferon gamma is increased in the T cell line after contacting the cell-based immunotherapy with an HDACi.

29. The method of claim 25 or 26, wherein cell-surface expression of CXCR3 is increased in the T cell line after contacting the cell-based immunotherapy with an HDACi.

30. The method of any one of claims 1 to 13, wherein the cell-based immunotherapy comprises a natural killer cell line or primary natural killer cell population.

31. The method of claim 30, wherein the natural killer cell line or population comprises a chimeric antigen receptor.

32. The method of claim 30, wherein the natural killer cell line or population comprises a high-affinity Fc receptor.

33. The method of any one of claims 30 to 32, wherein secretion of interferon gamma is increased in the natural killer cell line or population after contacting the cell-based immunotherapy with an HDACi.

34. The method of any one of claims 1 to 33, further comprising administering the cell-based immunotherapy to an individual afflicted with a disease.

35. The method of claim 34, wherein the cell-based immunotherapy is autologous to the individual afflicted with a disease.

36. The method of claim 34, wherein the disease is a cancer.

37. The method of claim 36, wherein the cancer is breast cancer, cervical cancer, colon cancer, head and neck cancer, kidney cancer, liver cancer, lung cancer, melanoma, ovarian cancer, pancreatic cancer, or prostate cancer.

38. The method of claim 36, wherein the cancer is a leukemia or lymphoma.

39. A method of adoptive cell immunotherapy comprising:
a) contacting a cell-based immunotherapy with an HDAC inhibitor (HDACi), wherein the HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide); and
b) administering the cell-based immunotherapy to an individual afflicted with a disease.

40. The method of claim 39, wherein contacting the cell-based immunotherapy with the HDACi is performed in vitro.

41. The method of claim 39, wherein the concentration of the HDACi is an amount sufficient to increase acetylation of histone H3.

42. The method of claim 41, wherein the concentration of the HDACi is less than about 1 micromolar.

43. The method of claim 41, is at least about 400 nanomolar.

44. The method of any one of claims 39 to 43, wherein the HDACi is contacted with the cell-based immunotherapy for at least 2 hours.

45. The method of any one of claims 39 to 43, wherein the HDACi is contacted with the cell-based immunotherapy for at least 16 hours.

46. The method of any one of claims 39 to 45, wherein the method comprises contacting the cell-based immunotherapy with interleukin-15.

47. The method of claim 46, wherein the interleukin-15 is contacted with the cell-based immunotherapy at a concentration of about 1 to about 100 ng/mL.

48. The method of claim 46, wherein the interleukin-15 is contacted with the cell-based immunotherapy at a concentration of about 5 to about 25 ng/mL.

49. The method of claim 46, wherein the interleukin-15 is contacted with the cell-based immunotherapy at a concentration of about 10 ng/mL.

50. The method of any one of claims 39 to 49, wherein the method comprises contacting the cell-based immunotherapy with a checkpoint inhibitor.

51. The method of claim 50, wherein the checkpoint inhibitor is an antibody that targets PDL-1 or PD-1.

52. The method of any one of claims 39 to 51, wherein the cell-based immunotherapy comprises a T-cell population.

53. The method of claim 52, wherein the T-cell population comprises a primary T-cell population derived from a healthy individual.

54. The method of claim 52, wherein the T-cell population comprises a primary T-cell population derived from an individual afflicted with a disease.

55. The method of claim 52, wherein the T-cell population comprises a primary T-cell population derived from the individual afflicted with the disease.

56. The method of any one of claims 52 to 55, wherein the T-cell population further comprises a chimeric antigen receptor (CAR).

57. The method of any one of claims 52 to 55, wherein the method further comprises stimulating the T-cell population with a tumor associated antigen.

58. The method of any one of claims 52 to 55, wherein the method further comprises stimulating the T-cell population with a pro-inflammatory cytokine.

59. The method of any one of claims 52 to 58, wherein the T-cell population is enriched for CD4 positive T cells.

60. The method of any one of claims 52 to 58, wherein the T-cell population is enriched for CD8 positive T cells.

61. The method of any one of claims 52 to 60, wherein FoxP3 expression is reduced in the T-cell population after contacting the cell-based immunotherapy with the HDACi.

62. The method of any one of claims 52 to 60, wherein secretion of interferon gamma is increased in the T-cell population after contacting the cell-based immunotherapy with the HDACi.

63. The method of any one of claims 52 to 60, wherein cell-surface expression of CXCR3 is increased in the T-cell population after contacting the cell-based immunotherapy with the HDACi.

64. The method of any one of claims 39 to 51, wherein the cell-based immunotherapy comprises a T-cell line.

65. The method of claim 64, wherein the T cell line comprises a chimeric antigen receptor.

66. The method of claim 64 or 65, wherein FoxP3 expression is reduced in the T cell line after contacting the cell-based immunotherapy with an HDACi.

67. The method of claim 64 or 65, wherein secretion of interferon gamma is increased in the T cell line after contacting the cell-based immunotherapy with an HDACi.

68. The method of claim 64 or 65, wherein cell-surface expression of CXCR3 is increased in the T cell line after contacting the cell-based immunotherapy with an HDACi.

69. The method of any one of claims 39 to 51, wherein the cell-based immunotherapy comprises a natural killer cell line or primary natural killer cell population.

70. The method of claim 69, wherein the natural killer cell line or population comprises a chimeric antigen receptor.

71. The method of claim 69, wherein the natural killer cell line or population comprises a high-affinity Fc receptor.

72. The method of any one of claims 69 to 71, wherein secretion of interferon gamma is increased in the natural killer cell line or population after contacting the cell-based immunotherapy with an HDACi.

73. The method of any one of claims 39 to 72, wherein the disease is a cancer.

74. The method of claim 73, wherein the cancer is breast cancer, cervical cancer, colon cancer, head and neck cancer, kidney cancer, liver cancer, lung cancer, melanoma, ovarian cancer, pancreatic cancer, or prostate cancer.

75. The method of claim 73, wherein the cancer is a leukemia or lymphoma.

76. A method of treating human immunodeficiency (HIV) infection in an individual comprising: administering to an individual with an HIV infection an effective amount of nanatinostat, wherein the individual with an HIV infection has an HIV viral load of less than 1000 copies of HIV RNA per milliliter of blood.

77. The method of claim 76, wherein the individual has an HIV viral load of less than 100 copies of HIV RNA per milliliter.

78. The method of claim 76 or 77, wherein nanatinostat is administered at a dose of less than 80 mg per day.

79. The method of claim 76 or 77, wherein nanatinostat is administered at a dose of less than 40 mg per day.

80. The method of claim 76 or 77, wherein nanatinostat is administered at a dose of less than 20 mg per day.

81. The method of any one of claims 76 to 80, further comprising administering an anti-HIV treatment to the individual with an HIV infection.

82. The method of claim 81, wherein the anti-HIV treatment comprises an anti-retroviral drug or pharmaceutically acceptable salt thereof.

83. The method of claim 82, wherein the anti-retroviral drug or pharmaceutically acceptable salt thereof is selected form the list consisting of Abacavir, Atazanavir, Darunavir, Dolutegravir, Efavirenz, Elvitegravir, Emtricitabine, Etravirine, Fosamprenavir, Lamivudine, Lopinavir, Maraviroc, Nevirapine, Raltegravir, Rilpivirine, Ritonavir, Tenofovir, Zidovudine, and combinations thereof.

84. The method of claim 81, wherein the anti-HIV treatment comprises an immunotherapy.

85. The method of claim 84, wherein the immunotherapy comprises an antibody that binds to an HIV polypeptide.

86. The method of claim 84, wherein the immunotherapy comprises a T-cell population.

87. The method of claim 86, wherein the T-cell population is transgenic for a chimeric antigen receptor specific for an HIV derived polypeptide.

88. The method of claim 86, wherein the T-cell population is a cytotoxic T cell population that specifically lyses HIV infected cells.

89. The method of claim 84, wherein the immunotherapy comprises a natural killer cell population.

90. The method of claim 89, wherein the natural killer cell population is transgenic for a chimeric antigen receptor specific for an HIV derived polypeptide.

91. The method of any one of claims 84 to 90 wherein the immunotherapy is contacted with a histone deacetylase inhibitor (HDACi) in vitro prior to administration to the individual with an HIV infection.

92. The method of claim 91, wherein the HDACi comprises nanatinostat, quisinostat (JNJ-26481585 (N-hydroxy-2-(4-((((1-methyl-1H-indol-3-yl)methyl)amino)methyl)piperidin-1-yl)pyrimidine-5-carboxamide)), R306465/JNJ-16241199 (N-hydroxy-5-(4-(naphthalen-2-ylsulfonyl)piperazin-1-yl)pyrimidine-2-carboxamide), Belinostat/PXD101, trichostatin A/TSA (7-[4-(dimethylamino)phenyl]-N-hydroxy-4,6-dimethyl-7-oxohepta-2,4-dienamide), ITF2357, CBHA, Givinostat/ITF2357, romidepsin, PCI-24781, depsipeptide (FR901228 or FK228), butyrate, phenylbutyrate, valproic acid, AN-9, CI-994, Entinostat/MS-275/SNDX-275, mocetinostat/MGCD0103 (N-(2-aminophenyl)-4-(4-pyridin-3-ylpyrimidin-2-ylamino)methyl)benzamide), m-carboxycinnamic acid, bishydroxamic acid, suberic bishydroxamic acid, oxamflatin, ABHA, SB-55629, pyroxamide, propenamides, aroyl pyrrolyl hydroxamides, or LAQ824 (((E)-N-hydroxy-3-[4-[[2-hydroxyethyl-[2-(1H-indol-3-yl)ethyl]amino]methyl]phenyl]prop-2-enamide), panobinostat/LBH-589, vorinsotat/SAHA, chidamide, or 4SC-202.

93. The method of claim 92, wherein the HDACi comprises nanatinostat.

94. The method of any one of claims 91 to 93, wherein the concentration of the HDACi is an amount sufficient to increase acetylation of histone H3.

95. The method of any one of claims 91 to 93, wherein the concentration of the HDACi is less than about 1 micromolar.

96. The method of any one of claims 91 to 93, wherein the HDACi is contacted with the immunotherapy for at least 2 hours.

97. The method of any one of claims 91 to 93, wherein the HDACi is contacted with the immunotherapy for at least 16 hours.

98. The method of anyone of claims 76 to 97, wherein the individual with an HIV infection has previously received an anti-HIV treatment.

99. The method of any one of claims 81 to 98, wherein the anti-HIV treatment is an anti-retroviral drug or pharmaceutically acceptable salt thereof.

100. A method for treating an individual with a latent viral infection comprising:
a) administering to an individual with the latent viral infection a first histone deactylase inhibitor (HDACi);
b) contacting a cell-based immunotherapy in vitro with a second HDACi, wherein the second HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide); and
c) administering the cell-based immunotherapy to the individual with the latent viral infection.

101. A method for treating an individual with a latent viral infection comprising:
a) administering to an individual with the latent viral infection a first histone deactylase inhibitor (HDACi), wherein the first HDACi comprises nanatinostat (2-(6-{[(6-Fluoroquinolin-2-yl)methyl]amino}-3-azabicyclo[3.1.0]hex-3-yl)-N-hydroxypyrimidine-5-carboxamide));
b) contacting a cell-based immunotherapy in vitro with a second HDACi; and
c) administering the cell-based immunotherapy to the individual with the latent viral infection.
